(19) United States
(12) Patent Application Publication
(10) Pub. No.: US 2021/0057053 A1
(43) Pub. Date: Feb. 25, 2021

(54) BRAIN HEALTH BASELINING

(71) Applicant: HealthTech Apps, Inc., Honolulu, HI (US)

(72) Inventors: Kyle Nainoa Manuma Chang, Honolulu, HI (US); Eleanor Noelani Foster, Honolulu, HI (US); Paul Keoni Newell, Honolulu, HI (US); Rick William Abelmann, Honolulu, HI (US)

(73) Assignee: HealthTech Apps, Inc., Honolulu, HI (US)

(21) Appl. No.: 17/045,748

(22) PCT Filed: Apr. 8, 2019
(86) PCT No.: PCT/US2019/026404

§ 371 Date: Oct. 6, 2020

Related U.S. Application Data
(60) Provisional application No. 62/654,175, filed on Apr. 6, 2018.

(51) Int. Cl. G16H 10/20; G16H 10/60; G16H 50/20; G16H 50/70; G16H 50/30; G06F 40/40

(52) U.S. Cl. CPC G16H 10/20; G16H 10/60; G16H 50/20; A61B 5/4064; G16H 50/30; G06F 40/40; G16H 50/70

(57) ABSTRACT

Disclosed is a method for establishing a brain health baseline from a brain health subject perspective and assessing brain health from the perspective of a healthcare provider or non-healthcare provider. Also disclosed is a system for establishing a brain health baseline from a brain health subject perspective and assessing brain health from the perspective of a healthcare provider or non-healthcare provider.

### BRIEF DESCRIPTION OF THE DRAWINGS

FIG. 1 depicts a diagram of an example of a system for brain health baselining.

FIG. 2 depicts a diagram of an example of an architecture including brain health baseline record managing system.

FIG. 3 depicts a diagram of an example of an architecture including a brain health subject system.

FIG. 4 depicts a diagram of an example of an architecture including a healthcare-provider subscriber system.

FIG. 5 depicts a diagram of an example of an architecture including a non-healthcare-provider subscriber system.

FIG. 6 depicts a flowchart of an example of a method for obtaining and providing a brain health baseline record of a subject.

FIG. 7 depicts a flowchart of an example of a method for generating a brain health baseline record of a subject.

FIG. 8 depicts a flowchart of an example of a method for obtaining a brain health baseline record of a brain health subject.

FIG. 9 depicts a flowchart of another example of a method for obtaining a brain health baseline record of a brain health subject.

FIG. 10 depicts a flowchart of an example of a method for brain health incident reporting and monitoring during recovery.

### DETAILED DESCRIPTION

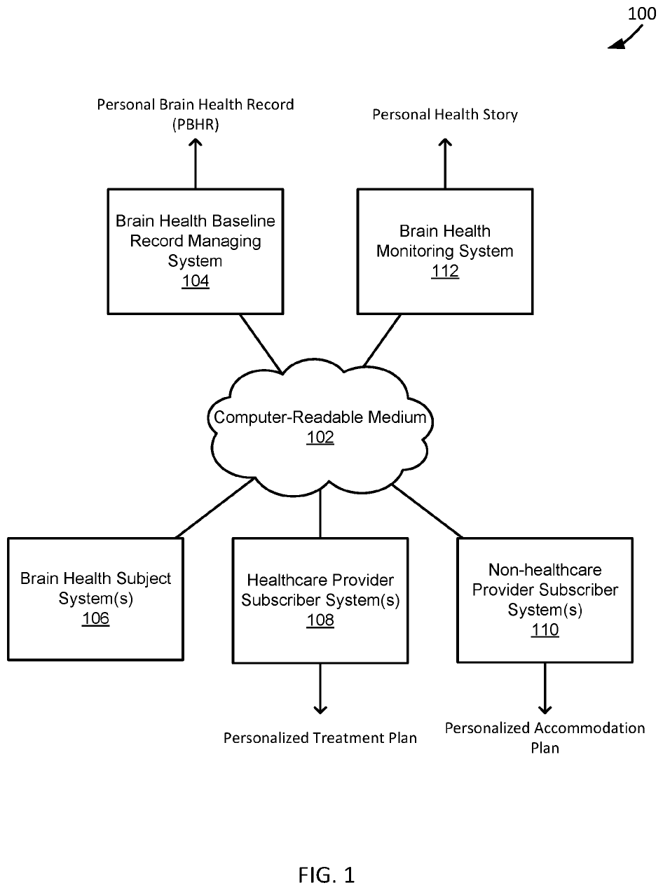

FIG. 1 depicts a diagram 100 of an example of a system for brain health baselining. The diagram 100 includes a computer-readable medium 102, a brain health baseline record managing system 104 coupled to the computer-readable medium 102, brain health subject system(s) 106 coupled to the computer-readable medium 102, healthcare-provider subscriber system(s) 108 coupled to the computer-readable medium 102, non-healthcare-provider subscriber system(s) 110 coupled to the computer-readable medium 102, and a brain health monitoring system 112 coupled to the computer-readable medium 102.

The computer-readable medium 102 is intended to represent a variety of potentially applicable technologies. For example, the computer-readable medium 102 can be used to form a network or part of a network. Where two components are co-located on a device, the computer-readable medium 102 can include a bus or other data conduit or plane. Where a first component is co-located on one device and a second component is located on a different device, the computer-readable medium 102 can include a wireless or wired back-end network or LAN. The computer-readable medium 102 can also encompass a relevant portion of a WAN or other network, if applicable.

As used in this paper, a “computer-readable medium” is intended to include all mediums that are statutory (e.g., in the United States, under 35 U.S.C. 101), and to specifically exclude all mediums that are non-statutory in nature to the extent that the exclusion is necessary for a claim that includes the computer-readable medium to be valid. Known statutory computer-readable mediums include hardware (e.g., registers, random access memory (RAM), non-volatile (NV) storage, to name a few), but may or may not be limited to hardware.

The computer-readable medium 102 and applicable systems, engines, devices, or the like coupled therewith can be implemented as a computer system, a plurality of computer systems, or parts of a computer system or a plurality of computer systems. In general, a computer system will include a processor, memory, non-volatile storage, and an interface and the examples described in this paper assume a stored program architecture, though that is not an explicit requirement of the machine. A typical computer system will usually include at least a processor, memory, and a device (e.g., a bus) coupling the memory to the processor. The processor can be, for example, a general-purpose central processing unit (CPU), such as a microprocessor, or a special-purpose processor, such as a microcontroller. A typical CPU includes a control unit, arithmetic logic unit (ALU), and memory (generally including a special group of memory cells called registers).

The memory can include, by way of example but not limitation, random access memory (RAM), such as dynamic RAM (DRAM) and static RAM (SRAM). The memory can be local, remote, or distributed. The bus can also couple the processor to non-volatile storage. The non-volatile storage is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software on the computer system. The non-volatile storage can be local, remote, or distributed. The non-volatile storage is optional because systems can be created with all applicable data available in memory.

In stored program architectures, software is typically stored in the non-volatile storage. Indeed, for large programs, it may not even be possible to store the entire program in the memory. Nevertheless, it should be understood that for software to run, if necessary, it is moved to a computer-readable location appropriate for processing, and for illustrative purposes, that location is referred to as the memory in this paper. Even when software is moved to the memory for execution, the processor will typically make use of hardware registers to store values associated with the software, and local cache that, ideally, serves to speed up execution. As used herein, a software program is assumed to be stored at an applicable known or convenient location (from non-volatile storage to hardware registers) when the software program is referred to as “implemented in a computer-readable storage medium.” A processor is considered to be “configured to execute a program” when at least one value associated with the program is stored in a register readable by the processor.

In one example of operation, a computer system can be controlled by operating system software, which is a software program that includes a file management system, such as a disk operating system. One example of operating system software with associated file management system software is the family of operating systems known as Windows® from Microsoft Corporation of Redmond, Wash., and their associated file management systems. Another example of operating system software with its associated file management system software is the Linux operating system and its associated file management system. The file management system is typically stored in the non-volatile storage and causes the processor to execute the various acts required by the operating system to input and output data and to store data in the memory, including storing files on the non-volatile storage.

The bus can also couple the processor to the interface. The interface can include one or more input and/or output (I/O) devices. The I/O devices can include, by way of example but not limitation, a keyboard, a mouse or other pointing device, disk drives, printers, a scanner, and other I/O devices, including a display device. The display device can include, by way of example but not limitation, a cathode ray tube (CRT), liquid crystal display (LCD), or some other applicable known or convenient display device. The interface can include one or more modem or network interface. It will be appreciated that a modem or network interface can be considered to be part of the computer system. The interface can include an analog modem, ISDN modem, cable modem, token ring interface, satellite transmission interface (e.g. “direct PC”), or other interfaces for coupling a computer system to other computer systems. Interfaces enable computer systems and other devices to be coupled together in a network.

The computer systems can be compatible with or implemented as part of or through a cloud-based computing system. As used in this paper, a cloud-based computing system is a system that provides virtualized computing resources, software and/or information to client devices. The computing resources, software and/or information can be virtualized by maintaining centralized services and resources that the edge devices can access over a communication interface, such as a network. “Cloud” may be a marketing term and for the purposes of this paper can include any of the networks described herein. The cloud-based computing system can involve a subscription for services or use a utility pricing model. Users can access the protocols of the cloud-based computing system through a web browser or other container application located on their client device.

A computer system can be implemented as an engine, as part of an engine, or through multiple engines. As used in this paper, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. The processor transforms data into new data using implemented data structures and methods, such as is described with reference to the FIGS. in this paper.

The engines described in this paper, or the engines through which the systems and devices described in this paper can be implemented, can be cloud-based engines. As used in this paper, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used in this paper, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a general- or specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described in this paper.

Datastores can include data structures. As used in this paper, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described in this paper, can be cloud-based datastores. A cloud-based datastore is a datastore that is compatible with cloud-based computing systems and engines.

Referring once again to the example of FIG. 1, the brain health baseline record managing system 104 is intended to represent hardware configured to provide a computer-based brain health baselining service. In a specific implementation, the brain health baseline record managing system 104 is configured to provide a dynamic brain health baselining questionnaires to the brain health subject system(s) 106. In a specific implementation, the dynamic brain health baselining questionnaire includes a prompt created using subject-specific attributes, which may be derived from subject-specific brain health baselining inputs, such as those provided by the subject, by healthcare providers familiar with the subject, by non-healthcare providers familiar with the subject, found on social networks of which the subject is a member, or other subject-specific sources. The brain health baseline record managing system 104 can use subject persona-specific sources of information to augment the effectiveness of the dynamic brain health baselining questionnaires, such as medical experimental sources, clinical sources, physiological sources, psychological sources, and other sources of information that provide insight regarding subjects having particular attributes, such as athlete, screenager, transgender person, or other attributes of the subject. The dynamic brain health baselining questionnaire presentation can also be subject- or profile-specific. For example, when a specific physical or mental state of a subject (e.g., lisping, mental illness, etc.) is known, the information can be considered in generating the dynamic brain health baselining questionnaire, such as by providing additional time to answer questions, more supportive language, or the like. The dynamic brain health baselining questionnaires can be responsive to known events, like a brain injury (e.g., concussion) or illness, which can be used to generate dynamic brain health baselining questionnaires administered over time to update previous baselines or create a historical baseline that is responsive to recovery milestones, including the time to reach or failure to reach expected recovery milestones.

It may be noted the term “questionnaire” is used throughout this paper, but it is not a requirement that the dynamic brain health baselining questionnaire actually include specific questions; while questions are likely, the goal of a dynamic brain health baselining questionnaire is to elicit a response, so an instruction (e.g., “Describe the emotion you are feeling”), silence during which a subject can do whatever they want, or just showing pictures or playing music can all be considered part of a dynamic brain health baselining questionnaire. The types of prompts provided in the dynamic brain health baselining questionnaires can range from simply a stimulus to elicit a response to prompts that, from subject responses, can signal cognitive capabilities, emotional stability, personality characteristics, or state of mind. Responses can be voluntary, such as by answering questions or following instructions, or non-voluntary, such as by having an increased heart rate or using subconscious body language. Instead of or in addition to dynamic questionnaires, the brain health baseline record managing system 104 can analyze data retrieved from the brain health subject system(s) 106 that is not responsive to a dynamic brain health baselining questionnaire, such as biometric data obtained through sensors, online behavior, technology use, or other data to which the brain health baseline record managing system 104 has permission to access.

In the example of FIG. 1, the brain health subject system(s) 106 are intended to represent one or more devices used by a brain health subject or agent thereof to capture a response to a dynamic brain health baselining questionnaire, which can be maintained at the brain health subject system(s) 106 and/or provided to the brain health baseline record managing system 104. A brain health subject is a person for whom a brain health baseline and monitoring record is created. The brain health subject need not have a known mental disorder or be at risk of any particular mental disorder. For example, brain health subjects could include athletes who may be at risk for a concussion, soldiers who may be exposed to combat stress, or teenagers who may or may not be at risk for bullying, depression, or risky behavior; or brain health subjects could include every athlete on a team, every soldier in a unit, or every teenager who volunteers to participate in a brain health baselining and monitoring program. Brain health subjects could also include people with known disorders, such as individuals who may behave erratically, become lethargic, or suffer some other symptom when not taking medication.

In a specific implementation, a brain health baseline record includes brain health baselining inputs provided by a brain health subject on a brain health subject system in response to one or more dynamic brain health baselining questionnaires and biometric data obtained from the brain health subject through sensors of the brain health subject system during the administration of the dynamic brain health baselining questionnaire. A brain health baseline can include, for example, a video of the brain health subject responding to a dynamic brain health baselining questionnaire. As an alternative to a video, a brain health baseline can include an audio baseline of a brain health subject, such as utterance made in response to a dynamic brain health baselining questionnaire, or a text baseline, such as text made in response to questions of a dynamic brain health baselining questionnaire. In an alternative, a brain health baseline is video, audio, or text of a brain health subject that is created in response to automated or opportunistic stimuli other than a dynamic brain health baselining questionnaire, and may, in some embodiments, be derived from media that was not explicitly created in response to a dynamic brain health baselining questionnaire or even for use as a brain health baseline (e.g., browsing history).

In a specific implementation, the content captured by the brain health subject system(s) 106 including biometric data obtained through sensors from brain health subject system(s) 106 are compiled, compressed, and communicated through the output of a personal brain health record (PBHR) or portion thereof at the brain health baseline record managing system 104. Alternatively, some or all of the PBHR could be compiled, compressed, and communicated at the brain health subject system(s) 106. For example, the brain health baseline record managing system 104 could be distributed across the brain health subject system(s) 106. In such an implementation, it may still be valuable to have a universal manager capable of taking advantage of personas of which multiple different brain health subjects are members for the purpose of improving dynamic brain health questionnaires and brain health in general, though such functionality could also be distributed.

In a specific implementation, the brain health subject system(s) 106 provide at least a subset of a PBHR to parties other than the subject. For example, emergency personnel could have a code that enables access to a brain health baseline record of a PBHR through a locked screen of a subject's smart phone, enabling first responders to determine whether slowness of speech, lack of dexterity, or the like that is currently exhibited by a subject deviates from baseline. In such an implementation, the brain health baseline record can include a “baseline” that is a multimedia file of the brain health subject responding to a dynamic brain health baselining questionnaire, which enables the first responders to compare the responses of the brain health subject to the baseline. Depending upon implementation-specific, configuration-specific, and other factors, a brain health baseline record could include other information than simply a baseline, such as a record of brain health history (e.g., concussion history), recovery milestone updates, or other medical information, such as blood type, allergies, prescriptions, or the like.

In a specific implementation, a locked screen of the brain health subject system(s) 106 include a bypass through which emergency personnel (or hospital staff) are permitted to access a baseline and other health records associated with a subject, such as current medications, allergies, blood type, or the like. The bypass can be implemented as a QR code or other optical code that takes appropriate parties to a site that includes brain health baseline records, and which can be protected by requiring two-factor authentication, such as a passcode provided by the brain health subject system(s) 106 and the first responders or other authorized parties. As one of many possible examples, a first responder could press a medical emergency button on a locked screen of a brain health subject's smartphone, which is part of the relevant one of the brain health subject system(s) 106. The brain health subject's locked screen could then display a code that could be used by an authorized subscriber system, which in this example would include the first responder, to obtain a passcode to unlock the brain health subject's smartphone, or at least a portion thereof, to obtain access to medical information stored on the device, or to access medical information at the brain health baseline record managing system 104. It is also possible the brain health subject would make a baseline and other medical information available to anyone who presses the medical emergency button on their locked screen. It may be noted the term brain health baseline record can also be used to refer to inputs to the brain health baseline record managing system 104 by the brain health subject, first responders, or other parties; whether the brain health baseline record actually includes a baseline is dependent upon context.

In the example of FIG. 1, the healthcare-provider subscriber system(s) 108 are intended to represent hardware configured to present a brain health baseline record to a healthcare provider subscriber or an agent thereof. In a specific implementation, the brain health baseline record for a brain health subject is accessed at the brain health baseline record managing system 104 or provided therefrom. Depending upon implementation-specific or other considerations, a healthcare provider may include a doctor, a nurse, some other healthcare provider, or an agent thereof. Depending upon implementation-specific or other considerations, the brain health baseline record may include visual, audio, and/or text input of a brain health subject generated in response to a dynamic brain health baselining questionnaire, brain health statistical data, and/or data of a subject profile of the brain health subject. In a specific implementation, the brain health baseline record is presented using an applicable system for presenting the questions and/or instructions, such as a 2D display, 3D display, a virtual reality (VR) enabled display, an augmented reality (AR) enabled display, an audio device (e.g., speaker), or the like. In a specific implementation, the applicable system for presenting the brain health baseline records is embodied in one or more portable devices such as a mobile phone, tablet, laptop, gaming device smart watch, smart speaker, hand-held medical devices, wearables, accessories, or the like, and/or one or more non-portable devices, such as a desktop computer, projector, smart television, medical devices, or the like.

In a specific implementation, the healthcare-provider subscriber system(s) 108 provide requests, monitoring instructions, and observations (in the form of a narrative, record, sensor data, or the like) to the brain health baseline record managing system 104 and dynamic brain health baselining questionnaires, which can depend upon the requests, monitoring instructions, and observations, to the brain health subject system(s) 106. Requests are made, for example, prior to an appointment with a brain health subject or when a brain health baseline record of a brain health subject is determined to be inadequate to formulate a personalized treatment plan of the brain health subject. Monitoring instructions are provided, for example, when a healthcare provider subscriber recommends a particular prompt for a next dynamic brain health baselining questionnaire or in accordance with recovery milestones following a diagnosis. Observations are provided, for example, when a diagnosis is non-conclusive in order to facilitate a shift in monitoring or when the value of the observational data can be used to further categorize a brain health subject into a persona.

In the example of FIG. 1, the non-healthcare-provider subscriber system(s) 110 are intended to represent hardware configured to present a brain health baseline record to a non-healthcare provider subscriber or an agent thereof. Depending upon implementation-specific or other considerations, a non-healthcare provider subscriber may include a sports team, athletic trainer, potential or current employer, a background investigating entity, a health insurance company, a government entity (e.g., DMV, veterans affair offices, etc.), an academic institute, an advertisement marketing company, or the like. In some embodiments, the brain health baseline record is similar to that described above with reference to the healthcare provider subscriber system(s) 108, with potentially audience-specific variation of data provided therein (e.g., to ensure HIPPA compliance).

In the example of FIG. 1, the brain health monitoring system 112 monitors a subject over time to create a personal health story. The brain health baseline record managing system 104 and the brain health monitoring system 112 can be considered part of the brain health baseline record managing system 104.

In an example of operation of the example system shown in FIG. 1, the brain health baseline record managing system 104 provides a dynamic brain health baselining questionnaire to brain health subject system(s) 106. The dynamic brain health baselining questionnaire may be unique to a brain health subject, in which case it would be sent to only one of the brain health subject system(s) 106, or unique to a persona, in which case it could be sent to more than one of the brain health subject system(s). Static brain health baselining questionnaires may or may not also be sent (e.g., when a potential brain health subject is being enrolled, but there is insufficient data about the subject to create a dynamic brain health baselining questionnaire). The brain health subject system(s) 106 present the dynamic brain health baselining questionnaire to brain health subject(s) and obtain brain health baselining inputs captured by the brain health subject system(s) 106 while the brain health subject(s) respond to the dynamic brain health baselining questionnaire(s). The brain health subject system(s) 106 generate brain health baseline record(s) using the brain health baselining inputs, and provide the brain health baseline record(s) to the brain health baseline record managing system 104. The brain health baseline record managing system 104 obtains the brain health baseline record(s), compiles a PBHR, and provides at least a subset of the PBHR to one or more of the healthcare-provider subscriber system(s) 108 and/or the non-healthcare-provider subscriber system(s) 110.

Advantageously, a system for obtaining and providing a brain health baseline record is capable of generating and providing a dynamic brain health baselining questionnaire to a brain health subject. The dynamic brain health baselining questionnaire presents profile-dependent prompts to the subject depending on the subject's attributes (e.g., gender, age, etc.) to facilitate a natural and/or informative response. Using voluntary and involuntary responses to the prompts, the system is capable of generating a PBHR, including a baseline that enables relevant parties to determine whether a person is currently deviating from the baseline (potentially signaling a health-related concern), attributes that enable the system to categorize brain health subjects into personas that may have different needs than other personas, and a historical record of brain health useful for generating a personal health story for the subject. Alternatively or in addition, the PBHR can be used to perform a brain health diagnosis of the subject or to assess the suitability of the subject for hiring, admission, deployment, or the like, speed up icebreakers by getting some advance information about the subject, and/or facilitate preparation of suitable accommodations for the subject, which may lead to saving time and cost for in-person inspection of the subject and saving lives in certain situations, such as first responders assessing the brain health of the subject.

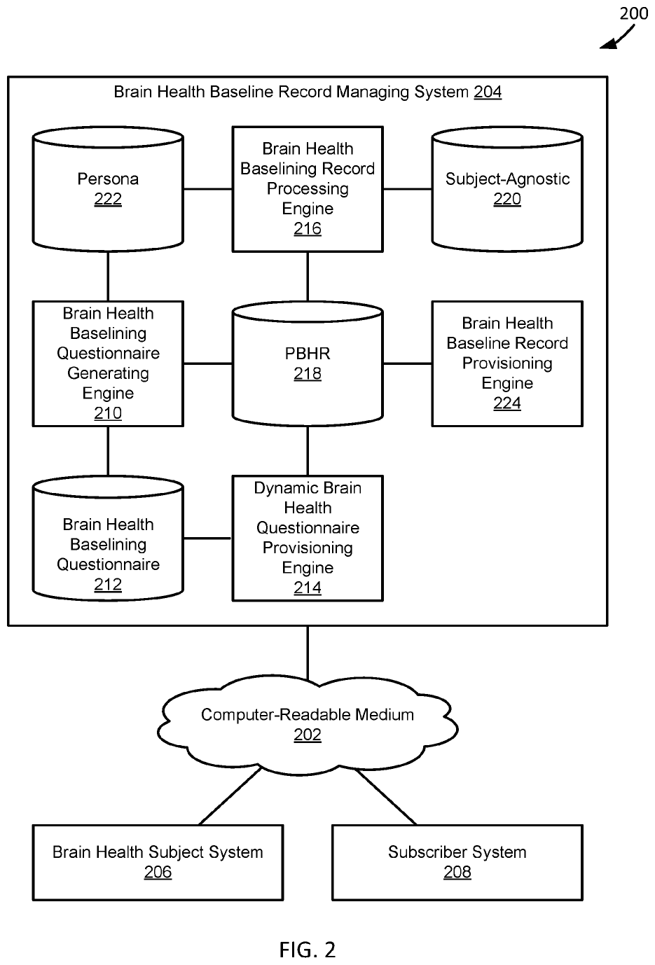

FIG. 2 depicts a diagram 200 of an example of an architecture including a brain health baseline record managing system. The diagram 200 includes a computer-readable medium 202, a brain health baseline record managing system 204 coupled to the computer-readable medium 202, a brain health subject system 206 coupled to the computer-readable medium 202, and a subscriber system 208 coupled to the computer-readable medium 202. In a specific implementation, the brain health baseline record managing system 204, the brain health subject system 206, and the subscriber system 208 correspond to the brain health baseline record managing system 104, one of the brain health subject system(s) 106, and one of the healthcare-provider subscriber system(s) 108 or one of the non-healthcare-provider subscriber system(s) 110 in FIG. 1, respectively.

In the example of FIG. 2, the brain health baseline record managing system 204 is intended to represent hardware configured to provide a dynamic brain health baselining questionnaire to the brain health subject system 206, use subject-specific data provided in response to the questionnaire and other data related to brain health to create a PBHR for each subject, and to share at least a subset of the PBHR with relevant parties, such as healthcare provider subscribers, non-healthcare provider subscribers, and non-subscribers (e.g., first responders who are not subscribers). In the diagram 200, the brain health baseline record managing system 204 includes a brain health baselining questionnaire generating engine 210, a brain health baselining questionnaire datastore 212, a dynamic brain health questionnaire provisioning engine 214, a brain health baselining record processing engine 216, a PBHR datastore 218, a subject-agnostic datastore 220, a persona datastore 222, and a brain health baseline record provisioning engine 224. The brain health baselining questionnaire generating engine 210, the brain health baselining questionnaire datastore 212, the dynamic brain health questionnaire provisioning engine 214, the brain health baselining record processing engine 216, the PBHR datastore 218, the subject-agnostic datastore 220, the persona datastore 222, and the brain health baseline record provisioning engine 224 are coupled, for example, for data communication.

In the example of FIG. 2, the brain health baselining questionnaire generating engine 210 is intended to represent hardware configured to generate a dynamic brain health baselining questionnaire, which is stored in the brain health baselining questionnaire datastore 212. In a specific implementation, a brain health baselining questionnaire is generated in accordance with a plurality of parameters for determining a resulting question for a specific brain health subject, referencing a PBHR of the specific brain health subject for that purpose, or in accordance with a plurality of parameters for determining a resulting question for a specific persona. Parameter values of the parameters may be modified through applicable machine learning techniques and stored in the persona datastore 222. For example, a first chain of questions is generated for first one or more brain health subjects having a first attribute (e.g., an attribute associated with a demographic (which, in this paper, is intended to include health/medical characteristics), geographic, psychographic, or behavioristic characteristic of a subject or group of subjects), and a second chain of questions is generated for second one or more brain health subjects having a second attribute. In another example, a first question is generated in response to a first response and/or reaction to a previous question, and a second question is generated for a second response and/or reaction to the previous question.

In the example of FIG. 2, the brain health baselining questionnaire datastore 212 is intended to represent a datastore configured to store one or more brain health baselining questionnaires including one or more brain health baselining questionnaires generated by the brain health baselining questionnaire generating engine 210. In a specific implementation, the brain health baselining questionnaire datastore 212 maintains the brain health baselining questionnaires in a brain health baselining questionnaire table including a plurality of entries each of which corresponds to a dynamic brain health baselining questionnaire. For example, an entry of the brain health baselining questionnaire table includes an identification of the brain health baselining questionnaire, profile-specific characteristics (demographic, geographic, psychographic, behavioristic, or other characteristics), parameter values associated with the brain health baselining questionnaire table, and stored location information of the dynamic brain health baselining questionnaire. The profile-specific characteristics enable the dynamic brain health questionnaire provisioning engine 214 to provide dynamic brain health baselining questionnaires depending upon characteristics of a target brain health subject. In a specific implementation, the brain health baselining questionnaire datastore 212 stores a machine learning model applicable to one or more brain health baselining questionnaires stored therein.

Depending upon implementation-specific or other considerations, a brain health baselining questionnaire is configured to facilitate dynamically provisioning thereof and responding thereto. For example, a brain health baselining questionnaire can have a field or metadata that restricts its provisioning to a particular time and receiving of a response to a time window to obtain a state of a brain health subject in the particular time window. Using a time window can encourage compliance with response window instructions. For example, a prompt related to sleep patterns may be provided in a dynamic brain health questionnaire only in the morning hours. As another example, a previously created questionnaire that can be reused with one or more subjects may only be provided to subjects of a particular gender (persona) and/or sent at a particular time, such as a birthday, homecoming, graduation, or the like (window).

In the example of FIG. 2, the dynamic brain health questionnaire provisioning engine 214 is intended to represent hardware configured to provide a dynamic brain health baselining questionnaire from the brain health baselining questionnaire datastore 212 to the brain health subject system 206 when a PBHR of a brain health subject associated with the brain health subject system 206 identifies a persona, or a timing, matching a defined persona, or a window, of the dynamic brain health baselining questionnaire. In a specific implementation, the dynamic brain health questionnaire provisioning engine 214 provides a dynamic brain health baselining questionnaire in a push message at a predetermined time or after a triggering event for brain health subjects identifiable as having a persona matching that of the dynamic brain health baselining questionnaire, or in advance of the predetermined time or (predicted) triggering event. For example, the dynamic brain health questionnaire provisioning engine 214 can provide a dynamic brain health baselining questionnaire at a particular point of time on a specific day or when the brain health subject system 206 first detects activity in a day (e.g., when a brain health subject activates a smart phone after waking up).

In a specific implementation, the dynamic brain health questionnaire provisioning engine 214 provides a dynamic brain health baselining questionnaire in a pull message in response to requests from the brain health subject system 206. For example, the dynamic brain health questionnaire provisioning engine 214 can receive a request for dynamic brain health baselining questionnaire from the brain health subject system 206 and send a dynamic brain health baselining questionnaire to the brain health subject system 206. In a specific implementation, the request for a dynamic brain health baselining questionnaire includes one or more profile attributes of a brain health subject associated with the brain health subject system 206, such as an identification (e.g., subject ID) of a subject. The dynamic brain health questionnaire provisioning engine 214 can obtain other profile attributes from the PBHR datastore 218 to enable provisioning of a dynamic brain health questionnaire. Attributes of a subject may include age, gender, weight, height, race, handedness, daily time spent on social media, pre-existing medical conditions (e.g., traumatic brain injury, attention deficit hyperactivity disorder (ADHD), disease, mental disorder, etc.), and residence (e.g., city, state, country, etc.), or other attributes obtainable from a PBHR associated with the brain health subject. In a specific implementation, when the dynamic brain health questionnaire provisioning engine 214 obtains a request for dynamic brain health baselining questionnaire from the brain health subject system 206, the dynamic brain health questionnaire provisioning engine 214 retrieves a brain health baselining questionnaire corresponding to profile attributes of the brain health subject from the brain health baselining questionnaire datastore 212 by referring to a brain health baselining questionnaire table stored in the brain health baselining questionnaire datastore 212. In a specific implementation, when the dynamic brain health questionnaire provisioning engine 214 retrieves a brain health baselining questionnaire corresponding to the request, the dynamic brain health questionnaire provisioning engine 214 provides the retrieved dynamic brain health baselining questionnaire to the brain health subject system 206.

Depending upon implementation-specific or other considerations, the timing of providing the dynamic brain health baselining questionnaire(s) may be when a subject registers for a brain health baselining service provided using the brain health baseline record managing system 204; at the time when a subject operates to obtain an appointment with a health provider through a computer-based brain health baselining service, which can be useful for a health provider in getting past routine questions or expediting an ice breaker based upon what is currently, historically, or potentially of interest to the brain health subject; or at some other specific time. Depending upon implementation-specific or other considerations, a dynamic brain health baselining questionnaire may be sent multiple times at occasionally, periodically, or at predetermined times and/or for different brain health subjects, and/or in advance of or after triggering events, such as appointments for a physical examination.

In the example of FIG. 2, the brain health baselining record processing engine 216 is intended to represent hardware configured to process a brain health baseline record obtained from an engine of the brain health subject system 206 responsive to a dynamic brain health baselining questionnaire provided to the brain health subject system 206, perform additional processing, and, if applicable, update a PBHR of the relevant subject in the PBHR datastore 218. Depending upon implementation-specific or other considerations, a brain health baseline record includes brain health baselining inputs made by a brain health subject in response to a dynamic brain health baselining questionnaire, such as a video, audio, text, or other memorialization of the brain health subject responding to the questionnaire along with biometric data obtained by the brain health subject system 206.

In a specific implementation, the brain health baseline record processing engine 216 is configured to generate brain health data statistics based on one or more brain health baseline records of multiple subjects. Depending upon implementation-specific or other considerations, the brain health data statistics may include average values or attributes associated with a brain health subject, group of brain health subjects, or brain health subject profile and average values or attributes associated with a control group. In a specific implementation, the brain health baseline record processing engine 216 is configured to add brain health baselining information obtained in association with a subject to a PBHR associated with the subject, which can result in a new brain health baseline for the subject. For example, when physiological data of the subject (e.g., body temperature, blood pressure, heart rate variability, blood type, etc.) is obtained from applicable sources including the brain health subject system 206 and the subscriber system 208, the brain health baseline record processing engine 216 adds the physiological data of the brain health subject to a PBHR for the brain health subject along with biometric data passively retrieved from the brain health subject system 206, which may indicate deviation from a baseline (or, equivalently, that a new baseline may be appropriate), that a baseline can be more precisely defined with new data, or the like.

In the example of FIG. 2, the PBHR datastore 218 is intended to represent a datastore configured to store PBHRs for brain health subjects, which can each include multiple brain health baseline records or data that can be used to generate brain health baseline records. In a specific implementation, the PBHR datastore 218 manages PBHRs using a brain health baseline record table including a plurality of entries each of which corresponds to a brain health baseline record. For example, an entry of the brain health baseline record table can include an identification of a subject, an identification of a brain health baseline record, an identification of one or more subscribers who are authorized to access the brain health baseline record, parameter values associated with the brain health baseline record table, and stored location information of brain health baseline record. In a specific implementation, the PBHR datastore 218 also stores a machine learning model applicable to one or more brain health baseline records stored therein, which can be occasionally, periodically, or continuously be used by the brain health baselining record processing engine 216 for improved accuracy or precision. The machine learning model is used to compute content by analyzing keywords and phrases recorded in the video/audio transcription to communicate what a brain health subject is reporting, in the form of a word art dashboard to display on a display device of the brain health subject system 206 and/or the subscriber system 208. For example, if the brain health subject system 206 captured video with these keywords: headache, blurred vision and light sensitivity, these words would be transmitted to the subject system and displayed as word art as a quick-glance summary of the subject's point-in-time response to a dynamic brain health questionnaire.

To the extent the brain health baselining questionnaire generating engine 210 personalizes data for a particular subject, the, e.g., demographic, characteristics of the subject can be obtained from the PBHR datastore 218, though there may be no data available for a new subject. In an implementation in which a questionnaire can be generated for anew subject (e.g., a subject for which insufficient data is known to enable customization of a questionnaire), a first questionnaire may include questions related to demographic, geographic, psychographic, and/or behavioristic characteristics, which can be maintained in the PBHR datastore 218 and used for future questionnaires. In an implementation in which the subject maintains his or her own PBHR, the PBHR datastore 218 can be implemented at the brain health subject system 206 and shared in part for the purpose of dynamic brain health questionnaire generation, and/or the brain health baselining questionnaire generating engine 210 can be implemented in whole or in part at the brain health subject system 206.

In the example of FIG. 2, the subject-agnostic datastore 220 is intended to represent a data from a medical experimental source, a clinical source, a physiological source, a psychological source, or a social network source, to name several possible sources. The sources are generally compilations or conclusions from studies that are applicable to a number of different subjects that can be grouped (as a persona) for brain health baselining, monitoring, or treatment purposes. Depending upon implementation-specific or other considerations, the data from subject-agnostic sources may be manually retrieved from the applicable sources or automatically retrieved therefrom in accordance with applicable triggering events, such as an update of clinical data or a brain injury incident.

In a specific implementation, the brain health baselining questionnaire generating engine 210 generates a dynamic brain health baselining questionnaire indirectly using the subject-agnostic datastore 220. What is meant by “indirectly” is the brain health baselining record processing engine 216 compares data from the PBHR datastore 218 (including data derived from the brain health subject system 208) with subject-agnostic data from the subject-agnostic datastore 220 to create personas comprising brain health subjects with characteristics that make organization into the persona useful from a brain health monitoring perspective, and updates the persona datastore 222 accordingly, which the brain health baselining questionnaire generating engine 210 then uses to generate a persona-specific dynamic brain health questionnaire. For example, when a specific physical or mental state of a subject (in the PBHR datastore 218) is known as a factor to find a specific mental disorder (from the subject-agnostic datastore 220), the information is taken into consideration in generating the dynamic brain health baselining questionnaire (for the relevant persona in the persona datastore 222).

In a specific implementation, the brain health baselining questionnaire generating engine 210 generates a brain health baselining questionnaire in a manner that depends upon brain health subject state, with or without the knowledge of a specific brain health subject. For example, the brain health baselining questionnaire generating engine 210 can generate a dynamic brain health baselining questionnaire for a persona.

In the example of FIG. 2, the brain health baseline record provisioning engine 224 is intended to represent an engine configured to provide a brain health baseline record to the subscriber system 208. In a specific implementation, the brain health baseline record provisioning engine 224 provides a brain health baseline record as a pull message in response to requests from a subscriber system 208. For example, the dynamic brain health questionnaire provisioning engine 214 can receive a request for brain health baseline record from the subscriber system 208 and send a brain health baseline record to the subscriber system 208. In a specific implementation, the request includes one or more attributes unique to a subscriber associated with the subscriber system 208 and to a PBHR, such as an identification (e.g., subscriber ID) of a subscriber, and an identification (e.g., subject ID) of a brain health subject for which a brain health baseline record is requested. In a specific implementation, when the brain health baseline record provisioning engine 224 receives a brain health baseline record request from the subscriber system 208, the brain health baseline record provisioning engine 224 retrieves a brain health baseline record corresponding to attributes included in the request from the PBHR datastore 218 by referencing a brain health baseline record table stored in the PBHR datastore 218. In a specific implementation, when the brain health baseline record provisioning engine 224 retrieves a brain health baseline record corresponding to the request, the brain health baseline record provisioning engine 224 provides the brain health baseline record to the subscriber system 208. Depending upon implementation-specific or other considerations, the timing of receiving a brain health baseline record request and providing the brain health baseline record may be at the time when a subscriber (e.g., a healthcare provider) records a future appointment with a brain health subject through a computer-based brain health baselining service of the brain health baseline record managing system 204, when a brain health subject is ready to be monitored or treated, or when a subject consents to provide the brain health baseline record to the subscriber system 208, to name a few.

In the example of FIG. 2, the brain health subject system 206 is intended to represent hardware configured to generate a brain health baseline record of the brain health subject using brain health baselining inputs made by the brain health subject in response to a dynamic brain health baselining questionnaire along with biometric data captured by the brain health subject system 206. In a specific implementation, an engine of the brain health subject system 206 captures video and/or audio of a brain health subject. For example, a video camera can be activated to determine whether a brain health client is asleep at a particular point in time in a day and, if so, to capture sleep states (e.g., snoring, mumbling, sweating, etc.) of the brain health subject. In another example, a microphone is activated to capture utterances made by a brain health subject. The specific physical or mental state of a brain health subject may include voluntary expressive features of a client such as a voiceprint of a subject, an utterance contents of a subject, a facial image of a subject, and a text input contents of a subject, and so on, and involuntary physical and biometric features of a subject such as body temperature, heart rate, eye tracking, blood pressure and so on. In a specific implementation, captured content is provided to the brain health baselining record processing engine 216 for inclusion in a PBHR of a brain health subject associated with the brain health subject system 206.

In a specific implementation, an engine of the brain health subject system 206 monitors an application (e.g., SNS, web browser, etc.) used on a device of the brain health subject system 206 and content associated therewith. In monitoring use of an application, in a specific implementation, an engine of the brain health subject system 206 may include a specific plug-in to applications and/or an operating system executable on the applicable device. For example, when an SNS application is monitored, an engine of the brain health subject system 206 creates a log (e.g., time and duration) of access to the SNS application, and captures screenshots of screens of the SNS application presented to the brain health client, which might lead to finding of causes (e.g., cyberbullying) of a mental state of a subject. In a specific implementation, when application monitoring is carried out, the identification of the application and/or contents thereof, such as the log and the screenshots, are provided to the brain health baselining record processing engine 216 for inclusion in a PBHR of a brain health subject associated with the brain health subject system 206.

In the example of FIG. 2, the subscriber system 208 is intended to represent hardware configured to obtain a brain health baseline record of a brain health subject from the brain health baseline record managing system 204 for use by a subscriber. In a specific implementation, an engine of the subscriber system 208 obtains a brain health baseline record of a brain health subject. What data is included in the brain health baseline record and how the brain health baseline record is used are context-specific. For example, non-medical persons may not have rights to view certain medical records, first responders might have a specific set of desired information (e.g., a baseline video, blood type, allergies, and drug prescriptions), and employers might be limited to data that does not identify the brain health subject as a protected class.

In an example of operation of the example system shown in FIG. 2, the brain health baselining questionnaire generating engine 210 generates a dynamic brain health baselining questionnaire referencing a PBHR in the PBHR datastore 218 and/or a persona in the persona datastore 222, and stores the dynamic brain health baselining questionnaire in the brain health baselining questionnaire datastore 212. The dynamic brain health questionnaire provisioning engine 214 provides a dynamic brain health baselining questionnaire to the brain health subject system 206. The brain health baselining record processing engine 216 obtains a brain health baseline record provided from the brain health subject system 206 and, if applicable, updates a PBHR associated with the brain health subject system 206 in the PBHR datastore 218 and/or uses the brain health baseline record and data from the subject-agnostic datastore 220 to update a persona associated with the brain health subject system 206 in the persona datastore 222. The brain health baseline record provisioning engine 224 receives a request for a brain health baseline record for a brain health subscriber associated with the brain health subject system 206 from the subscriber system 208 and provides a brain health baseline record to the subscriber system 210 from (or derived from data in) the PBHR datastore 218.

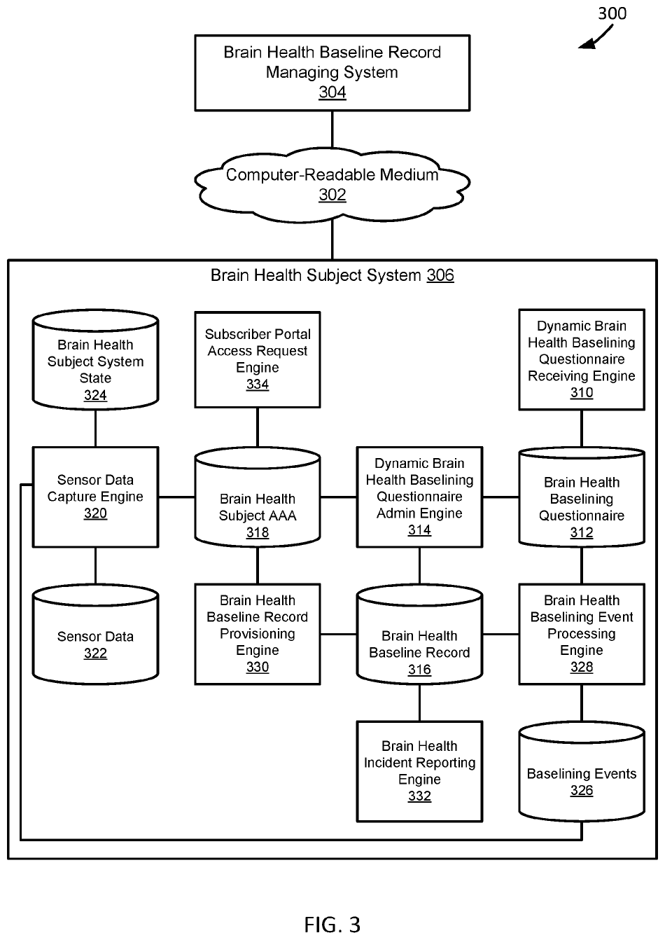

FIG. 3 depicts a diagram 300 of an example of an architecture including a brain health subject system. The diagram 300 includes a computer-readable medium 302, a brain health baseline record managing system 304 coupled to the computer-readable medium 302, and a brain health subject system 306 coupled to the computer-readable medium 302. In a specific implementation, the brain health baseline record managing system 304 and the brain health subject system 306 correspond to the brain health baseline record managing system 104 and one of the brain health subject system(s) 106 in FIG. 1, respectively, and/or the brain health baseline record managing system 204 and the brain health subject system 206 in FIG. 2, respectively.

In the diagram 300, the brain health subject system 306 includes a dynamic brain health baselining questionnaire receiving engine 310, a brain health baselining questionnaire datastore 312, a dynamic brain health baselining questionnaire admin engine 314, a brain health baseline record datastore 316, a brain health subject AAA datastore 318, a sensor data capture engine 320, a sensor data datastore 322, a brain health subject system state datastore 324, a baselining events datastore 326, a brain health baselining events processing engine 326, a brain health baseline record provisioning engine 330, a brain health incident reporting engine 330, and a subscriber portal access request engine 334. In a specific implementation, the dynamic brain health baselining questionnaire receiving engine 310, the brain health baselining questionnaire datastore 312, the dynamic brain health baselining questionnaire admin engine 314, the brain health baseline record datastore 316, the brain health subject AAA datastore 318, the sensor data capture engine 320, the sensor data datastore 322, the brain health subject system state datastore 324, the baselining events datastore 326, the brain health baselining event processing engine 328, the brain health baseline record provisioning engine 330, the brain health incident reporting engine 330, and the subscriber portal access request engine 334 are coupled, for example, for data communication.

In the example of FIG. 3, the dynamic brain health baselining questionnaire receiving engine 310 is intended to represent hardware configured to communicate with the brain health baseline record managing system 304 to obtain a dynamic brain health baselining questionnaire for provisioning to a brain health subject or an agent thereof. In a specific implementation in which the dynamic brain health baselining questionnaire receiving engine 310 uses a pull message to obtain dynamic brain health baselining questionnaires, the dynamic brain health baselining questionnaire receiving engine 310 sends a request for a dynamic brain health baselining questionnaire to a dynamic brain health questionnaire provisioning engine of the brain health baseline record managing system 304 and receives the dynamic brain health baselining questionnaire in response to the request. The pull message can include an identification of a brain health subject, which provides brain health baseline record managing system 304 data sufficient to access a PBHR of the brain health subject and provide a dynamic brain health baselining questionnaire that is suitable for a persona of the brain health subject. Alternatively or in addition, the pull message can include profile information, such as a self-reported potential brain injury, which the brain health baseline record managing system 304 uses to identify a persona of the brain health subject (e.g., a person who potentially has a brain injury) and provide a dynamic brain health baselining questionnaire that is suitable for that persona.

In a specific implementation in which the brain health baseline record managing system 304 uses a push message to provide a dynamic brain health baselining questionnaire to the dynamic brain health baselining questionnaire receiving engine 310, the brain health baseline record managing system 304 sends a dynamic brain health baselining questionnaire in accordance with a default delivery schedule, a persona-dependent delivery schedule (e.g., patients of a particular physician and/or patients that have particular symptoms or risk factors), a profile-dependent delivery schedule (e.g., brain health subject preferences), event-dependent delivery schedule (e.g., after a self-reported potential brain injury). A schedule can include a one-time delivery, occasional (potentially time-variable) delivery, periodic delivery, and can change as circumstances warrant.

The dynamic brain health baselining questionnaire receiving engine 310 stores the dynamic brain health baselining questionnaire in the brain health baselining questionnaire datastore 312 for administration to a brain health subject at a specific time or within a specific time window and/or when a brain health subject matches persona-specific requirements of the dynamic brain health baselining questionnaire. One example of a persona is a brain health subject has an incomplete profile, which can occur when a brain health subject is initially enrolled in a brain health baselining and/or monitoring service or when a subscriber enrolls a brain health subject, but does not provide adequate, ideal, or optional profile data. Another example of a persona is a brain health subject is a member of a particular high school football team. Another example of a persona is a brain health subject who is at a particular milestone of a brain injury recovery period.

In the example of FIG. 3, the brain health baselining questionnaire datastore 312 is intended to represent a datastore configured to store one or more dynamic brain health baselining questionnaires. In a specific implementation, the brain health baselining questionnaire datastore 312 maintains the dynamic brain health baselining questionnaires using a dynamic brain health baselining table in a similar manner as the brain health baselining questionnaire datastore 212 in FIG. 2.

In the example of FIG. 3, the dynamic brain health baselining questionnaire admin engine 314 is intended to represent hardware configured to present to a subject or agent thereof one or more questions, instructions, or other prompts based on a dynamic brain health baselining questionnaire stored in the brain health baselining questionnaire datastore 312. The dynamic brain health baselining questionnaire can be presented in accordance with parameters of the dynamic brain health baselining questionnaire (e.g., during a specified time window) and may include matching parameters of the dynamic brain health baselining questionnaire with parameters of a brain health baseline record in the brain health baseline record datastore 316 (e.g., when the dynamic brain health baselining questionnaire is for a particular persona and the brain health baseline record includes a value indicative of the particular persona for a brain health subject). In a specific implementation, the dynamic brain health baselining questionnaire admin engine 314 consults the brain health subject authentication, authorization, and accounting (AAA) datastore 318 for permission before administering a dynamic brain health baselining questionnaire, capturing responses to prompts of the dynamic brain health baselining questionnaire, accepting responses to prompts (e.g., a brain health subject may be able to redo), or approving responses.

The prompts of the dynamic brain health baselining questionnaire trigger a verbal response, a text response, and/or a somatic response (e.g., gesture or eye movement), which may be voluntary or involuntary (e.g., biometric). The prompts may include a question, an instruction to perform a particular action, an instruction not to perform a particular action, a puzzle, free association images, or the like, and could even simply be music or an image that is used to capture a somatic response. Depending upon implementation-specific or other considerations, the dynamic brain health baselining questionnaire admin engine 314 presents a dynamic brain health baselining questionnaire within a specific time window, such as when a subject registers for a brain health baselining service provided through the brain health baseline record managing system 304, or when a subject acts to obtain a doctor appointment through a brain health baselining service. In a specific implementation, the dynamic brain health baselining questionnaire is presented using an applicable system for presenting the dynamic brain health baselining questionnaire, such as a 2D display, 3D display, a virtual reality (VR) enabled display, an augmented reality (AR) enabled display, an audio device (e.g., speaker), and so on. In a specific implementation, the applicable system for presenting the dynamic brain health baselining questionnaire may be embodied in portable devices such as a mobile phone, tablet, laptop, gaming device smart watch, smart speaker, wearables, accessories, equipment, and so on, non-portable devices, such as a desktop computer, projector, smart television, and so on, and/or medical devices.

In the example of FIG. 3, the brain health baseline record datastore 316 is intended to represent a datastore configured to store one or more brain health baseline records including one or more brain health baseline records, a subset of which (the subset potentially including all of the brain health baseline records) can be referred to as a PBHR for a brain health subject. In a specific implementation, the brain health baseline record datastore 316 maintains brain health baseline records using a brain health baseline record table in a similar manner as the brain health baseline record datastore 216 in FIG. 2. The amount of information in the brain health baseline record datastore 316 may be more than (at least for a particular brain health subject), the same as (subject to synchronization delays), or less than that maintained by the brain health baseline record managing system 304, and the brain health baseline record datastore 316 could be shared with the brain health baseline record managing system 304 in, for example, a cloud-based implementation.

As was mentioned previously, in a specific implementation, the dynamic brain health baselining questionnaire admin engine 314 can access the brain health baseline record datastore 316 to assist in the dynamic administration of brain health baselining questionnaires. The brain health baseline record datastore 316 may initially be seeded with data from subscribers (potentially via the brain health baseline record managing system 304) or start empty (potentially with a first questionnaire including some standard questions to obtain basic information, such as name, date of birth, and the like). Over time, the brain health baseline record 316 can be augmented by subscribers and/or the brain health baseline record managing system 304, but, in a specific implementation, will also be updated by the brain health baselining event processing engine 328 and/or the brain health incident reporting engine 330, described later. The brain health baseline record provisioning engine 330, also described later, shares contents of the brain health baseline record datastore 316 with appropriate parties.

In the example of FIG. 3, the brain health subject AAA datastore 318 is intended to represent a datastore configured to store brain health subject access instructions, preferences, and permissions. In a specific implementation, the dynamic brain health baselining questionnaire admin engine 314 is only permitted to administer a questionnaire in accordance with preferences in the brain health subject AAA datastore 318. For example, a brain health subject can set a “do not disturb” flag, have a preference for no questionnaires at a given time period, or the like. In a specific implementation, the dynamic brain health baselining questionnaire admin engine 314 queries the brain health subject AAA datastore 318 at various times during administration of a dynamic brain health baselining questionnaire, such as when asking whether access to a camera is permitted, and the AAA responses indicate whether the sensor data capture engine 320 is allowed to operate during a particular time frame and/or allow updating of the baselining events datastore 326.

In the example of FIG. 3, the sensor data capture engine 320 is intended to represent hardware sensors and input devices and, if necessary, interfaces through which sensor data can be collected. configured to enable a subject to memorialize a baseline. In an implementation in which the baseline includes a video file, the sensor data capture engine 320 includes a camera and an audio recording device. Where the baseline includes biometric data, the sensor data capture engine 320 can include other input devices and/or passive capture mechanisms that retrieve biometric data. Depending upon implementation-specific or other considerations, sensor data can be generated by an applicable device, such as an image sensor, a microphone, a motion detector, or the like, and stored in the sensor data datastore 322. For example, when an image sensor generates image data (e.g., still and/or video image data) of a subject while the subject is administered a dynamic brain health baselining questionnaire, the sensor data capture engine 320 obtains the image data and applies a timestamp or other data associated with brain health subject system state from the brain health subject system state datastore 324 (which may include a timestamp applied to an image by a camera). For illustrative purposes, the sensor data capture engine 320 does not limit data capture to a questionnaire response window, but the brain health subject AAA datastore 318 may include a limitation on what data is indicated to be part of the baselining events datastore 326, such as by limiting data capture to the questionnaire response window. For example, the brain health baselining event processing engine 328 could obtain data occasionally, periodically, continuously, or only during a questionnaire response data capture window. Advantageously, a brain health subject or an agent thereof may maintain sensor data and baselining events separately and include only that sensor data that is explicitly indicated to be appropriate for consideration to belong, or to be used to determine what should belong, in the brain health baseline record datastore 316, in accordance with rules or preferences.

In the example of FIG. 3, the sensor data datastore 322 is intended to represent a datastore configured to store input from sensors and input devices. The input can be limited in accordance with the brain health subject AAA datastore 318. Thus, the data in the sensor data datastore 322 may be restricted to particular applications, users, or the like. For illustrative purposes, the sensor data datastore 322 is intended to represent essentially all data devices of the brain health subject system 306 obtains, regardless of use, rapid age-out, or other features of the data.

In the example of FIG. 3, the brain health subject system state datastore 324 is intended to represent a datastore configured to maintain device state for one or more devices of the brain health subject system 306, such as a clock. The sensor data capture engine 328 can combine content (e.g., a video of a brain health subject) from the sensor data datastore 322 with an indication of brain health subject system state (e.g., a timestamp of the video) from the brain health subject system state datastore 324. The sensor data capture engine 320, when authorized to do so in accordance with the brain health subject AAA datastore 318, includes (e.g., stores or, if not explicitly storing, indicates inclusion of) content from the sensor data datastore 322, with or without augmentation using the brain health subject system state datastore 324, in the baselining events datastore 326.

In the example of FIG. 3, the baselining events datastore 326 is intended to represent a datastore of baselining events, such as video recordings, images, text, online activity, electronic device use, calendar events, or the like that the brain health baselining event processing engine 328 can use to create, read, update, or delete (CRUD) the brain health baseline record datastore 316. Depending upon the type of baselining event, the baselining event may be considered a brain health baseline record, considered part of a brain health baseline record, or be used to create, update, or delete a brain health baseline record. For example, a video can be characterized as a brain health baseline record, a timestamp may be considered part of a brain health baseline record, and specific biometric readings, such as a calendar entry may be omitted, but used explain a missed questionnaire (e.g., “brain health subject may have been on vacation”).

In the example of FIG. 3, the brain health baselining event processing engine 328 is intended to represent hardware configured to use baselining events from the baselining events datastore 326 to CRUD the brain health baseline record datastore 316. An example of creating a brain health baseline record is storing a video as a new brain health baseline record. An example of reading a brain health baseline record is comparing a prior brain health baseline record with a new baselining event to determine whether a recovery milestone appears to have been met (and either creating a new brain health baseline record using the data, or updating a brain health baseline record using the data). An example of updating a brain health baseline record is adding a new amount of time to an online activity summary. An example of deleting a brain health baseline record is determining a high risk category is no longer applicable (e.g., football season is over). In a specific implementation, historical data is retained in order to enable the provisioning of a personal health story with a complete history.

In a specific implementation, by CRUDing the brain health baseline record datastore 316, the brain health baselining event processing engine 328 modifies a PBHR that includes demographic, geographic, psychographic, and/or behavioristic characteristics, based on captured subject-specific data. PBHRs, which are generally unique to a subject, can be conceptually grouped at the brain health baseline record managing system 304 into what may be referred to as a persona (a “type” of subject). Personas can be treated similarly (e.g., a first questionnaire or first portion of a questionnaire can be used for each subject having a first persona and a second questionnaire or second portion of a questionnaire can be used for each subject having a second persona). Any given brain health subject may have a unique or non-unique combination of personas. In implementations in which the personas are human-readable types, it is more likely brain health subjects will have non-unique combinations of personas compared to systems that use machine learning to correlate characteristics to define a potentially extremely large set of possible personas. In a specific implementation, when a subject profile is generated, the brain health baselining event processing engine 328 stores one or more brain health baseline records defining the subject profile in the brain health baseline record datastore 316, which can at least conceptually be considered part of a PBHR.

In a specific implementation, the brain health baselining event processing engine 328 processes baselining events in the baselining events datastore 326 to identify key phrases useful for assessing brain health state. Depending upon brain health state, a brain health subject may be categorized into a new persona, which can trigger different dynamic brain health baselining questionnaire prompts, healthcare provider alerts, or the like. Key words can be derived from text, whether entered by the brain health subject or an agent thereof, transcribed from audio of the brain health subject or an agent thereof, generated to define brain health state derived from audio of the brain health subject (e.g., tone, cadence, volume, or the like), generated to define brain health state derived from video of the brain health subject (e.g., gestures, eye tracking data, hyperactivity, or the like), generated to define biometric sensor readings, or the like. The brain health baselining event processing engine 328 can store a word cloud of key phrases as a brain health baseline record in the brain health baseline record datastore 316. Alternatively or in addition, the brain health baseline record provisioning engine 330 could generate key phrases from brain health baseline records in the brain health baseline record datastore 316 and display them as a word cloud. Key phrases or combinations of key phrases can also serve to identify a new persona for a brain health subject, resulting in different brain health baselining questionnaires and/or administration schedules (e.g., in accordance with a recovery period).

In the example of FIG. 3, the brain health baseline record provisioning engine 330 is intended to represent hardware configured to provide a brain health baseline record from the brain health baseline record datastore 316 to an authorized party. For example, the brain health baseline record provisioning engine 330 can provide a brain health baseline record from the brain health baseline record datastore 316 to the brain health baseline record managing system 304.

In a specific implementation in which the brain health baseline record provisioning engine 330 uses a pull message to provide brain health baseline records, a brain health baseline record processing engine of the brain health baseline record managing system 304 sends a request for a brain health baseline record to the brain health baseline record provisioning engine 330 and the brain health baseline record provisioning engine 330 provides a brain health baseline record from the brain health baseline record datastore 316 in response to the request. The pull message can include an identification of the brain health baseline record managing system 304 and the brain health baseline record includes an identification of the applicable brain health subject (or a socket or packet header includes identifying information). Alternatively or in addition, the pull message can come from subscribers or other parties, in which case the brain health baseline record provisioning engine 330 can access the brain health subject AAA datastore 318 to determine whether a request is from a source authorized to receive a brain health baseline record, whether some of a brain health baseline record should be redacted, or the like.

In a specific implementation in which the brain health baseline record provisioning engine 330 uses a push message to provide a brain health baseline record to a brain health baseline record processing engine of the brain health baseline record managing system 304, the brain health baseline record provisioning engine 330 sends a brain health baseline record in accordance with a synchronization schedule (e.g., after a brain health record is created, updated, or deleted), a default delivery schedule, a persona-dependent delivery schedule (e.g., patients of a particular physician and/or patients that have particular symptoms or risk factors), a profile-dependent delivery schedule (e.g., brain health subject preferences), event-dependent delivery schedule (e.g., after a self-reported potential brain injury). A schedule can include a one-time delivery, occasional (potentially time-variable) delivery, periodic delivery, and can change as circumstances warrant. The brain health baseline record managing system 304 may or may not be responsible for updating other parties; in a specific implementation, the brain health baseline record provisioning engine 330 updates all or a subset of relevant parties. The brain health subject AAA datastore 318 can be used by the brain health baseline record provisioning engine 330 to ensure data provided to other systems is in accordance with brain health subject privacy rules.

In the example of FIG. 3, the brain health incident reporting engine 332 is intended to represent hardware configured to facilitate reporting by a brain health subject of brain health incidents. For example, a smartphone of the brain health subject system 306 may display a button indicating “I got rocked,” which a brain health subject can press after experiencing a potentially harmful incident. The brain health incident reporting engine 332 updates the brain health baseline record 316, which, depending upon implementation- and/or configuration-specific factors, can trigger the brain health baseline record provisioning engine 330 to update the brain health baseline record managing system 304 and/or other parties, which, in turn, can provide a dynamic brain health baselining questionnaire responsive to the reported brain health incident. The dynamic brain health baselining questionnaire may also be pre-stored in the brain health baselining questionnaire datastore 312 in anticipation of an applicable brain health incident to enable the dynamic brain health baselining questionnaire admin engine 314 to administer the dynamic brain health baselining questionnaire more quickly. In a specific implementation, the brain health incident reporting engine 332 is activated in response to a predefined event, such as when a biometric reading indicates blood sugar is dangerously low, a sensor on a football helmet detects a dangerously strong impact, or the like.

In the example of FIG. 3, the subscriber portal access request engine 334 is intended to represent hardware configured to provide access to data on the brain health subject system 306 or the brain health baseline record managing system 304 to authorized parties. In a specific implementation, the subscriber portal access request engine 334 includes a locked screen interface. For example, a smartphone of the brain health subject system 306 can have a locked screen with a “Medical Information” button (potentially with more or less descriptive information depending upon how well-known the feature is known to first responders).

In a relatively straight-forward implementation, a brain health baseline record suitable for public display can be provided through the subscriber portal access request engine 334. For example, when a “Medical Information” button is pressed, a smartphone can remain locked, but begin playing a brain health baseline video. A window may also display information such as blood type, prescription medications, allergies, or the like. In this way, some information can be made available to anyone who presses the button, not just subscribers.

In a somewhat more complex implementation, a brain health subject identifier can be provided on a locked screen of a smartphone that is part of the brain health subject system. By making a subject identifier available at the brain health subject system 306, first responders, or other relevant parties, can access relevant brain health baseline records, from at least one of the brain health baseline records datastore 316 and a PBHR datastore of the brain health medical baseline record managing system 304, depending upon implementation- and/or configuration-specific factors.

In a specific implementation, the subscriber portal access request engine 334 presents a visual code symbol from the brain health subject AAA datastore 318. Depending upon implementation-specific or other considerations, a visual code symbol presented by the subscriber portal access request engine 334 may be employed for categorizing a brain health subject (e.g., by blood type), authenticating a brain health subject (e.g., by confirming membership in a brain health baselining and monitoring service), and identifying a brain health subject (e.g., by name and social security number). In alternative implementations, the visual code symbol can be presented using a 3D display, a virtual reality (VR) enabled display, an augmented reality (AR) enabled display, and so on. The brain health subject code need not be visible; for example, the code could be provided via an RFID system for display on a subscriber device. By selecting (e.g., activating a QR code), entering (e.g., typing in a brain health subject text-based code), or otherwise using a brain health subject code, a subscriber can login to a brain health baseline and monitoring service to access a brain health baseline record obtained from the brain health baseline record managing system 304 through a portable devices such as a mobile phone, tablet, laptop, gaming device smart watch, smart speaker, or the like, non-portable devices, such as a desktop computer, projector, smart television, or the like, and/or medical devices. The brain health baseline record managing system 304 can make use of two-factor authentication, the login of the subscriber and the public brain health subject code, to ensure the subscriber is authorized to access the brain health baseline records. The brain health subject code need not be static; it could cycle through different codes. Alternatively or in addition, a subscriber could use a brain health subject code to send a message to the brain health baseline record managing system 304 to unlock the smartphone of the brain health subject or, more likely if the smartphone manufacturers are not compliant, display brain health baseline record information on a locked screen.

Depending upon implementation-specific or other considerations, an applicable system for presenting a brain health baselining questionnaire and/or a brain health subject code, such as a 2D display, 3D display, a virtual reality (VR) enabled display, an augmented reality (AR) enabled display, may also be configured to present a live consultation screen for communicating with applicable subscribers, such as a doctor, a nurse, or other healthcare provider or agent thereof, which enables a brain health subject or first responder and a healthcare provider to communicate without meeting in person if an in-person meeting is deemed unnecessary or for a preliminary communication, such as when rushing to a hospital. Alternatively or in addition, the subscriber portal access request engine 334 may trigger a notification to a hospital or alerts to other interested parties when a first responder activates the brain health subject code, in order to have medical information available in advance of arrival.

In an example of operation of the example system shown in FIG. 3, the dynamic brain health baselining questionnaire receiving engine 310 obtains a dynamic brain health baselining questionnaire from the brain health baseline record managing system 304, and stores the dynamic brain health baselining questionnaire in the brain health baselining questionnaire datastore 312. The dynamic brain health baselining questionnaire admin engine 314 presents a dynamic brain health baselining questionnaire from the brain health baselining questionnaire datastore 312 to a brain health subject or agent thereof in accordance with matching characteristics of a brain health subject in the brain health baseline record datastore 316 and rules from the brain health subject AAA datastore. The sensor data capture engine 320 stores captured data in the sensor data datastore 322 and, if applicable, augments the captured data using the brain health subject system state datastore 324, then, if permitted in accordance with the brain health subject AAA datastore 318, stores baselining events derived from the sensor data and brain health subject system state in the baselining events datastore 326. The brain health baselining event processing engine 328 uses baselining events obtained from the brain health subject, an agent thereof, or other parties associated therewith, to generate a brain health baseline record for storage in the brain health baseline record datastore 316, which may change the applicability of a brain health baselining questionnaire in the brain health baselining questionnaire datastore 312. The brain health baseline record provisioning engine 330 transmits a brain health baseline record stored in the brain health baseline record datastore 316 to the brain health baseline record managing system 304. Later, the brain health incident reporting engine 332 may update the brain health baseline record 316 to indicate a brain health incident has occurred, potentially causing the dynamic brain health baselining questionnaire admin engine 314 to administer an applicable brain health baselining questionnaire in response to the brain health incident. Also later, the subscriber portal access request engine 334 may trigger the brain health baseline record provisioning engine 330 to provide a brain health baseline record from the brain health baseline record datastore 316 to a party that is authorized using the brain health subject AAA datastore 318.

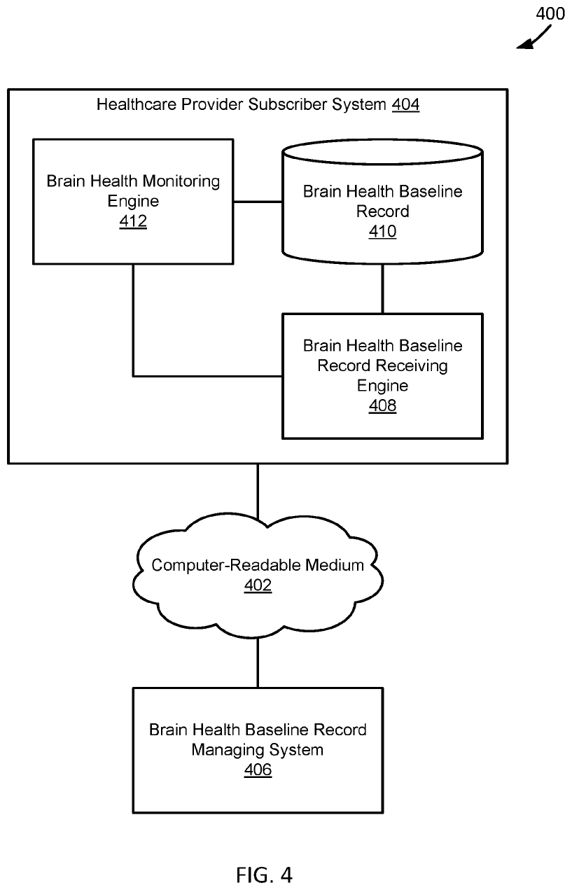

FIG. 4 depicts a diagram 400 of an example of an architecture including a healthcare-provider subscriber system. The diagram 400 includes a computer-readable medium 402, a healthcare provider subscriber system 404 coupled to the computer-readable medium 402, and a brain health baseline record management system 406 coupled to the computer-readable medium 402. In a specific implementation, the healthcare-provider subscriber system 404 and the brain health baseline record management system 406 correspond to one of the healthcare provider subscriber system(s) 108 and the brain health baseline record management system 104 in FIG. 1, respectively.

The healthcare provider subscriber system 404 is intended to represent hardware configured to provide a request for brain health baseline record for a brain health subject to the brain health baseline record management system 406, obtain the requested brain health baseline record from the brain health baseline record management system 406, and present the brain health baseline record for brain health diagnosis of the brain health subject. The brain health baseline record management system 406 is intended to represent hardware configured to provide a brain health baseline record for a brain health subject to the healthcare-provider subscriber system 404, in response to a request for the brain health baseline record from the healthcare-provider subscriber system 404.

The healthcare-provider subscriber system 404 includes a brain health baseline record receiving engine 408, a brain health baseline record datastore 410, and a brain health monitoring engine 412. In a specific implementation, the brain health baseline record receiving engine 408, the brain health baseline record datastore 410, and the brain health monitoring engine 412 are coupled, for example, for data communication.

The brain health baseline record receiving engine 408 is intended to represent hardware configured to communicate with the brain health baseline record managing system 406 to obtain a brain health baseline record. In a specific implementation, in obtaining a brain health baseline record, the brain health baseline record managing system 406 provides a request for brain health baseline record to the brain health baseline record managing system 406, and receives the brain health baseline record in response thereto. Depending upon implementation-specific or other considerations, the brain health baseline record managing system 406 provides the request for brain health baseline record at applicable timing, such as timing when a healthcare-provider subscriber obtains a doctor appointment for a brain health subject from an applicable system such as the brain health baseline record managing system 406, or when a brain health subject consents to disclose a brain health baseline record to the healthcare-provider subscriber. In a specific implementation, upon receiving a brain health baseline record, the brain health baseline record receiving engine 408 stores the brain health baseline record in the brain health baseline record datastore 410.

The brain health baseline record datastore 410 is intended to represent a datastore configured to store one or more brain health baseline records including one or more brain health baseline records obtained by the brain health baseline record receiving engine 408. In a specific implementation, the brain health baseline record datastore 410 maintains the stored brain health baseline records using a brain health baseline record table in a similar manner as the brain health baseline record datastore 216 in FIG. 2 and/or the brain health baseline record datastore 316 in FIG. 3.

The brain health monitoring engine 412 is intended to represent an engine configured to perform processing based on one or more brain health baseline records stored in the brain health baseline record datastore 410. In a specific implementation, the brain health monitoring engine 412 is configured to present one or more brain health baseline records stored in the brain health baseline record datastore 410. Depending upon implementation-specific or other considerations, the one or more brain health baseline records may include visual, audio, and/or text input of a brain health subject generated in response to a dynamic brain health baselining questionnaire presented to the brain health subject, brain health statistic data, and/or data of a subject profile of the brain health subject along with biometric data retrieved from the subject system. In a specific implementation, the one or more brain health baseline records are presented using an applicable system for presenting the questions and/or instructions, such as a 2D display, 3D display, a virtual reality (VR) enabled display, an augmented reality (AR) enabled display, an audio device (e.g., speaker), and so on. In a specific implementation, the applicable system for presenting the brain health baseline records may be embodied in portable devices such as a mobile phone, tablet, laptop, gaming device smart watch, smart speaker, and so on, non-portable devices, such as a desktop computer, projector, smart television, wearables, accessories, equipment, Internet of Things (IoT) sensor devices, and so on, and/or medical devices.

In a specific implementation, the brain health monitoring engine 412 is configured to obtain an additional monitoring instruction made by a healthcare-provider subscriber and provide the additional monitoring instruction to the brain health baseline record managing system 406, such that an additional dynamic brain health baselining questionnaire based on the additional monitoring instruction is sent to a brain health subject system (e.g., one of the brain health subject system(s) 106 in FIG. 1). It may be noted the healthcare provider subscriber system 404 may include a questionnaire datastore (not shown) or can request another system, such as a brain health baseline record managing system to provide a questionnaire (see, e.g., the brain health baseline record managing system 204 in FIG. 2). Depending upon implementation-specific or other considerations, an additional monitoring instruction may be made when a brain health baseline record of a brain health subject is not compliant, does not include sufficient records to make a brain health personalized treatment plan of the brain health subject, or when a healthcare-provider subscriber decides to provide a stimulus and/or a treatment to a brain health subject in accordance with a brain health diagnosis of the brain health subject based on the brain health baseline record. In a specific implementation, the additional dynamic brain health baselining questionnaire may be personalized to the brain health subject profile or persona and/or the healthcare provider. For example, a portion of the additional dynamic brain health baselining questionnaire may be generated in the voice of the healthcare provider (e.g., physician) or a mood-setting image can be matched to a geographic characteristic of the subject.

In a specific implementation, the brain health monitoring engine 412 is configured to obtain data associated with a subject from data entry by a healthcare provider or agent thereof, readings from medical equipment or other sensors, and create or update a brain health baseline record in the brain health baseline record datastore 410. The brain health monitoring engine 412 can also generate or facilitate in the generation of a brain health personalized treatment plan for a brain health subject based on inputs made by a healthcare-provider subscriber in accordance with a brain health baseline record for the brain health subject, and store the brain health personalized treatment plan in applicable datastore, such as the brain health baseline record datastore 410. A diagnosis or personal treatment plan can, at least conceptually, be considered part of the brain health baseline record. Depending upon implementation-specific or other considerations, the brain health monitoring engine 412 is configured to enable the brain health diagnosis to be accessible by applicable entities, such as the brain health subject, other healthcare-provider subscribers, and non-healthcare-provider subscribers, depending on consent of the brain health subject and authorization of the healthcare-provider subscriber that created the brain health diagnosis and personalized treatment plan.

In a specific implementation, the brain health monitoring engine 412 is configured to cause an applicable system, such as a 2D display, 3D display, a virtual reality (VR) enabled display, an augmented reality (AR) enabled display, to present a live consultation screen for communicating with applicable brain health subjects, which enables the brain health subject and a healthcare-provider subscriber can communicate without meeting in person.

In an example of operation of the example system shown in FIG. 4, the brain health baseline record receiving engine 408 provides a request for brain health baseline record for a subject to the brain health baseline record managing system 406 and obtains the requested brain health baseline record for the subject from the brain health baseline record managing system 406. The brain health baseline record receiving engine 408 stores the requested brain health baseline record in the brain health baseline record datastore 410. The brain health monitoring engine 412 causes the brain health baseline record for the subject to be presented for brain health diagnosis, formulation of a personalized treatment plan, and processes additional monitoring instructions made by a healthcare provider, if any. The brain health baseline record receiving engine 408 provides the additional monitoring instructions to the brain health baseline record managing system 406 for forward to a corresponding health care subject system.

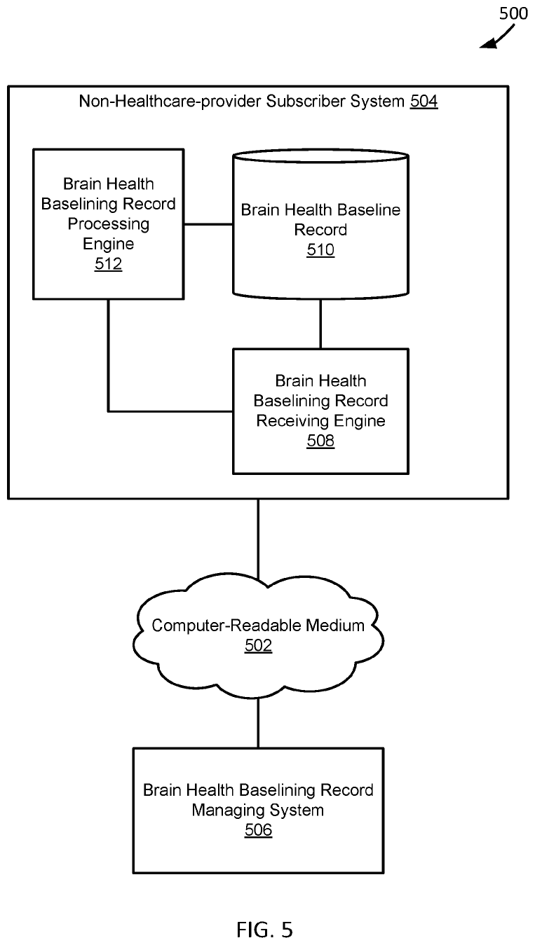

FIG. 5 depicts a diagram 500 of an example of an architecture including a non-healthcare-provider subscriber system. The diagram 500 includes a computer-readable medium 502, a non-healthcare-provider subscriber system 504 coupled to the computer-readable medium 502, and a brain health baseline record management system 506 coupled to the computer-readable medium 502. In a specific implementation, the non-healthcare-provider subscriber system 504 and the brain health baseline record management system 506 correspond to one of the non-healthcare-provider subscriber system(s) 110 and the brain health baseline record management system 104 in FIG. 1, respectively. In a specific implementation, the non-healthcare-provider subscriber system 504 functions in a similar manner as the healthcare-provider subscriber system 404 in FIG. 4.

The non-healthcare-provider subscriber system 504 is intended to represent hardware configured to provide a request for brain health baseline record for a brain health subject to the brain health baseline record management system 506, obtain the requested brain health baseline record from the brain health baseline record management system 506, and present the brain health baseline record for various applicable purposes, such as investigation, inspection, screening, examination, and/or monitoring of the brain health subject. The brain health baseline record management system 506 is intended to represent hardware configured to provide a brain health baseline record for a brain health subject to the non-healthcare-provider subscriber system 504, in response to a request for the brain health baseline record from the non-healthcare-provider subscriber system 504.

The non-healthcare-provider subscriber system 504 includes a brain health baseline record receiving engine 508, a brain health baseline record datastore 510, and a brain health baseline record processing engine 512. In a specific implementation, the brain health baseline record receiving engine 508, the brain health baseline record datastore 510, and the brain health baseline record processing engine 512 are coupled, for example, for data communication.

The brain health baseline record receiving engine 508 is intended to represent hardware configured to communicate with the brain health baseline record managing system 506 to obtain a brain health baseline record, PBHR, personalized health story, and/or personalized treatment plan. In a specific implementation, in obtaining a brain health baseline record, the brain health baseline record managing system 506 provides a request for brain health baseline record to the brain health baseline record managing system 506, and receives the brain health baseline record in response thereto. Depending upon implementation-specific or other considerations, the brain health baseline record managing system 506 provides the request for brain health baseline record at applicable time, such as when a brain health subject consents to disclose a brain health baseline record to the non-healthcare-provider subscriber. In a specific implementation, upon receiving a brain health baseline record, the brain health baseline record receiving engine 508 stores the brain health baseline record in the brain health baseline record datastore 510.

The brain health baseline record datastore 510 is intended to represent a datastore configured to store one or more brain health baseline records including one or more brain health baseline records obtained by the brain health baseline record receiving engine 508. In a specific implementation, in storing brain health baseline records, the brain health baseline record datastore 510 manages the stored brain health baseline records using a brain health baseline record table in a similar manner as the brain health baseline record datastore 410 in FIG. 4.

The brain health baseline record processing engine 512 is intended to represent an engine configured to perform processing based on one or more brain health baseline records stored in the brain health baseline record datastore 510. In a specific implementation, the brain health baseline record processing engine 512 is configured to present one or more brain health baseline records stored in the brain health baseline record datastore 510. Depending upon implementation-specific or other considerations, the one or more brain health baseline records may include visual, audio, and/or text input of a brain health subject generated in response to a dynamic brain health baselining questionnaire presented to the brain health subject, brain health statistic data, and/or data of a subject profile of the brain health subject and biometric data retrieved from the subject system. In a specific implementation, the one or more brain health baseline records are presented using an applicable system for presenting the questions and/or instructions, such as a 2D display, 3D display, a virtual reality (VR) enabled display, an augmented reality (AR) enabled display, an audio device (e.g., speaker), and so on. In a specific implementation, the applicable system for presenting the brain health baseline records may be embodied in portable devices such as a mobile phone, tablet, laptop, gaming device smart watch, smart speaker, and so on, non-portable devices, such as a desktop computer, projector, smart television, wearables, accessories, equipment, IoT sensor, and so on, and/or medical devices. In a specific implementation, a brain health baseline record presented by the brain health baseline record processing engine 512 may be a redacted version and part of the brain health baseline record may be concealed, depending on an access privilege level of the non-healthcare-provider subscriber.

In a specific implementation, the brain health baseline record processing engine 512 is configured to obtain an additional monitoring request made by a non-healthcare-provider subscriber and provide the additional monitoring request to the brain health baseline record managing system 506, such that an additional dynamic brain health baselining questionnaire based on the additional monitoring request is sent to a brain health subject system (e.g., one of the brain health subject system(s) 106 in FIG. 1). Depending upon implementation-specific or other considerations, an additional monitoring request may be made when a brain health baseline record of a brain health subject does not include sufficient records for make decisions about a brain health of the brain health subject.

In an example of operation of the example system shown in FIG. 5, the brain health baseline record receiving engine 508 provides a request for brain health baseline record for a subject to the brain health baseline record managing system 506 and obtains the requested brain health baseline record for the subject from the brain health baseline record managing system 508. The brain health baseline record receiving engine 508 stores the requested brain health baseline record in the brain health baseline record datastore 510. The brain health baseline record processing engine 512 causes the brain health baseline record for the subject to be presented to a non-healthcare-provider subscriber, and processes additional monitoring requests made by the non-healthcare-provider subscriber, if any. The brain health baseline record receiving engine 508 provides the additional monitoring requests to the brain health baseline record managing system 506 for forward to a corresponding health care subject system.

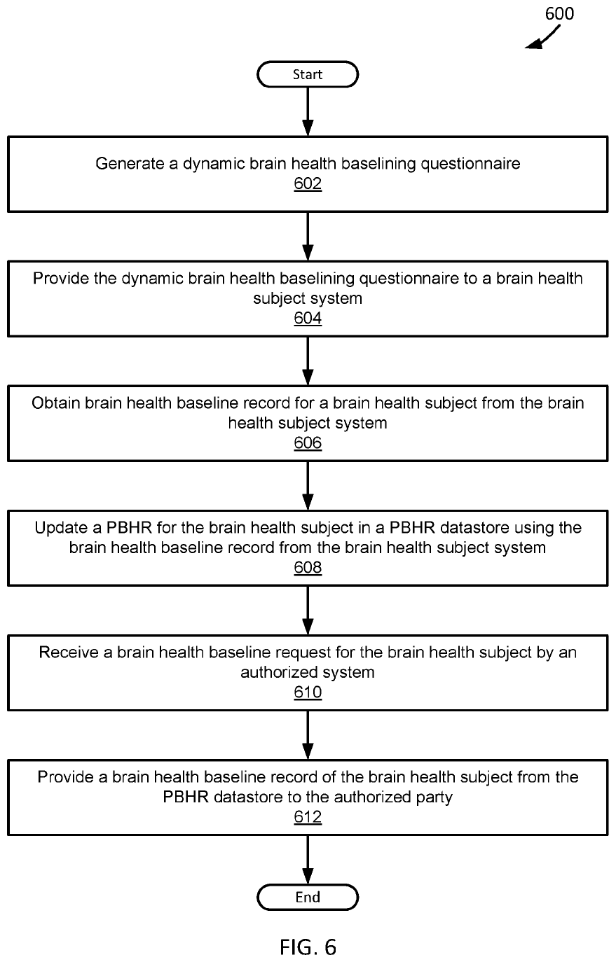

FIG. 6 depicts a flowchart 600 of an example of a method for obtaining and providing a brain health baseline record of a subject. This flowchart and the other flowcharts described in this paper illustrate modules (and potentially decision points) organized in a fashion that is conducive to understanding. It should be recognized, however, that the modules can be reorganized for parallel execution, reordered, modified (changed, removed, or augmented), where circumstances permit. The flowchart 600 begins at module 602 with generating a dynamic brain health baselining questionnaire. An applicable engine such as the brain health baselining questionnaire generating engine 210 in FIG. 2 generates the dynamic brain health baselining questionnaire.

The flowchart 600 continues to module 604 with providing the dynamic brain health baselining questionnaire to a brain health subject system. An applicable engine such as the dynamic brain health questionnaire provisioning engine 214 in FIG. 2 provides the dynamic brain health baselining questionnaire to the brain health subject system.

The flowchart 600 continues to module 606 with obtaining brain health baseline record for a brain health subject from the brain health subject system. An applicable engine such as the brain health baselining record processing engine 216 in FIG. 2 obtains the brain health baseline record generated by a brain health subject system.

The flowchart 600 continues to module 608 with updating a PBHR for the brain health subject in a PBHR datastore using the brain health baseline record from the brain health subject system. An applicable datastore such as the PBHR datastore 218 in FIG. 2 is updated when a brain health baseline record is received.

The flowchart 600 continues to module 610 with receiving a brain health baseline request from an authorized system. An applicable engine such as the brain health baseline record provisioning engine 224 in FIG. 2 obtains the brain health baseline request.

The flowchart 600 continues to module 612 with providing a brain health baseline record of the brain health subject from the PBHR datastore to the authorized party. An applicable engine such as the brain health baseline record provisioning engine 224 in FIG. 2 provides the brain health baseline record. The subscriber system 208 in FIG. 2 is an example of an authorized party.

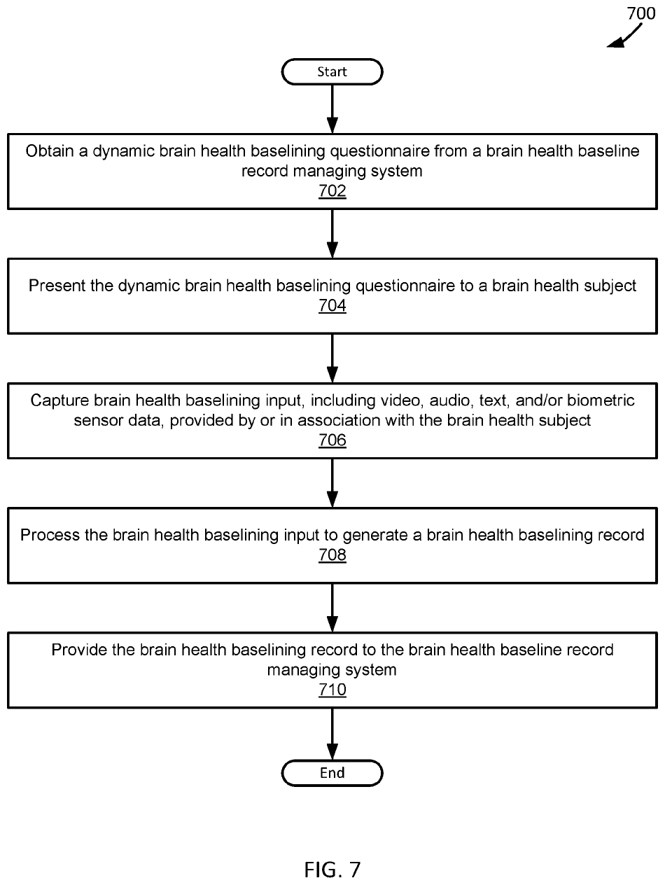

FIG. 7 depicts a flowchart 700 of an example of a method for generating a brain health baseline record of a subject, including video, audio, text, and/or biometric sensor data. The flowchart 700 begins at module 702 with obtaining a dynamic brain health baselining questionnaire from a brain health baseline record managing system. An applicable engine such as the dynamic brain health baselining questionnaire receiving engine 310 in FIG. 3 obtains the dynamic brain health baselining questionnaire from the brain health baseline record managing system. The brain health baseline record managing system 304 of FIG. 3 is an example of an applicable brain health baseline record managing system.

The flowchart 700 continues to module 704 with presenting a dynamic brain health baselining questionnaire to a brain health subject. An applicable engine such as the dynamic brain health baselining questionnaire admin engine 314 in FIG. 3 presents the dynamic brain health baselining questionnaire to the brain health subject.

The flowchart 700 continues to module 706 with capturing brain health baselining input, including video, audio, text, and/or biometric sensor data, provided by or in association with the brain health subject. An applicable engine such as the sensor data capture engine 320 in FIG. 3 captures the brain health baselining input, including video, audio, text, and/or biometric sensor data, provided by or in association with the brain health subject. Data may also be provided via a subscriber system, such as the healthcare provider subscriber system(s) 108 or the non-healthcare provider subscriber system(s) 110 of FIG. 1.

The flowchart 700 continues to module 708 with processing brain health baselining input to generate brain health baseline record. An applicable engine such as the brain health baselining event processing engine 328 in FIG. 3 processes the brain health baselining input to generate the brain health baseline record.

The flowchart 700 ends to module 710 with providing the brain health baseline record to the brain health baseline record managing system. An applicable engine such as the brain health baseline record provisioning engine 330 in FIG. 3 provides the brain health baseline record to the brain health baseline record managing system.

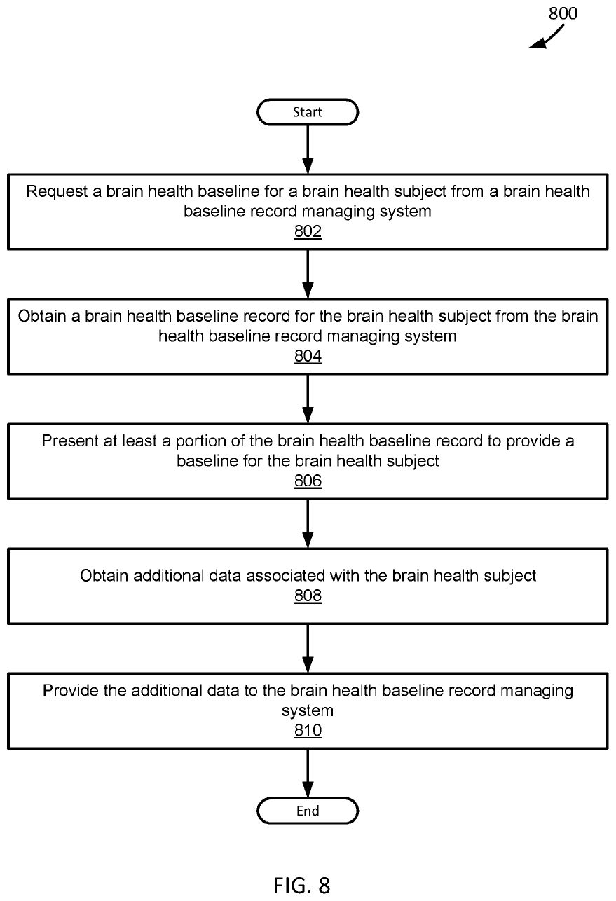

FIG. 8 depicts a flowchart 800 of an example of a method for obtaining a brain health baseline record of a brain health subject. The flowchart 800 begins at module 802 with requesting a brain health baseline for a brain health subject from a brain health baseline record managing system. An applicable engine such as the brain health record receiving engine 412 in FIG. 4 causes the request for brain health baseline record for a brain health subject to be sent to the brain health baseline record managing system (e.g., the brain health baseline record managing system 406 in FIG. 4).

The flowchart 800 continues to module 804 with obtaining a brain health baseline record for the brain health subject from the brain health baseline record managing system. An applicable engine such as the brain health baseline record receiving engine 408 in FIG. 4 obtains the brain health baseline record for the subject from a brain health baseline record managing system (e.g., the brain health baseline record managing system 406 in FIG. 4) and stores the brain health baseline record in applicable datastore such as the brain health baseline record datastore 410 in FIG. 4.

The flowchart 800 continues to module 806 with presenting at least a portion of the brain health baseline record to provide a baseline for the brain health subject. An applicable engine such as the brain health monitoring engine 412 in FIG. 4 causes at least a portion of the brain health baseline record to be presented on an applicable device, such as a display. A baseline can be a pre-episode of injury, pre-illness, or simply an earlier snapshot of the brain health subject that can be used for the purpose of comparison with the brain health subject at present.

The flowchart 800 continues to module 808 with obtaining additional data associated with the brain health subject. An applicable engine such as the brain health monitoring engine 412 in FIG. 4 obtains additional data associated with the brain health subject via an applicable user interface, such as a keyboard, microphone, video recorder, medical device, wearable, or the like. The additional data can be entered by a healthcare provider, a non-healthcare provider, or some other interested party.

The flowchart 800 ends at module 810 with providing the additional data to the brain health baseline record managing system. An applicable engine such as the brain health baseline record receiving engine 408 in FIG. 4 provides additional data to a brain health baseline record managing system (e.g., the brain health baseline record managing system 406 in FIG. 4).

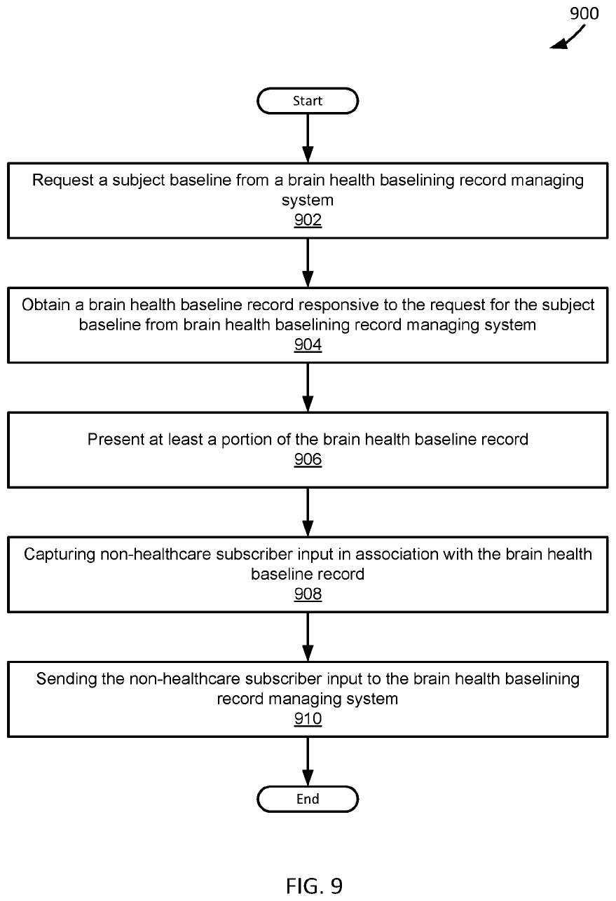

FIG. 9 depicts a flowchart 900 of another example of a method for obtaining a brain health baseline record of a subject. The flowchart 900 begins at module 902 with requesting a subject baseline from a brain health baseline record managing system. An applicable engine such as the brain health baseline record receiving engine 508 in FIG. 5 requests a subject baseline from a brain health baseline record managing system (e.g., the brain health baseline record managing system 506 in FIG. 5) and stores the brain health baseline record in applicable datastore such as the brain health baseline record datastore 510 in FIG. 5.

The flowchart 900 continues to module 904 with obtaining a brain health baseline record responsive to the request for the subject baseline from a brain health baseline record managing system. An applicable engine such as the brain health baseline record receiving engine 508 in FIG. 5 obtains the brain health baseline record responsive to the request for the subject baseline from the brain health baseline record managing system (e.g., the brain health baseline record managing system 506 in FIG. 5) and stores the brain health baseline record in an applicable datastore such as the brain health baseline record datastore 510 in FIG. 5.

The flowchart 900 continues to module 906 with presenting at least a portion of the brain health baseline record. An applicable engine such as the brain health baseline record processing engine 512 in FIG. 5 causes the brain health baseline record for the subject to be presented on an applicable device, such as a display. In an embodiment, the brain health baseline record for the subject is presented for investigation, inspection, screening, examination, and/or monitoring of the subject.

The flowchart 900 continues to module 908 with capturing non-healthcare subscriber input in association with the brain health baseline record. An applicable engine such as the brain health baseline record processing engine 512 in FIG. 5 processes the non-healthcare subscriber input captured in association with the brain health baseline record by an applicable user interface, such as a keyboard, microphone, wearable, or the like.

The flowchart 900 ends at module 910 with sending the non-healthcare subscriber input to the brain health baselining record managing system. An applicable engine such as the brain health baseline record receiving engine 508 in FIG. 5 provides non-healthcare subscriber input to a brain health baseline record managing system (e.g., the brain health baseline record managing system 506 in FIG. 5).

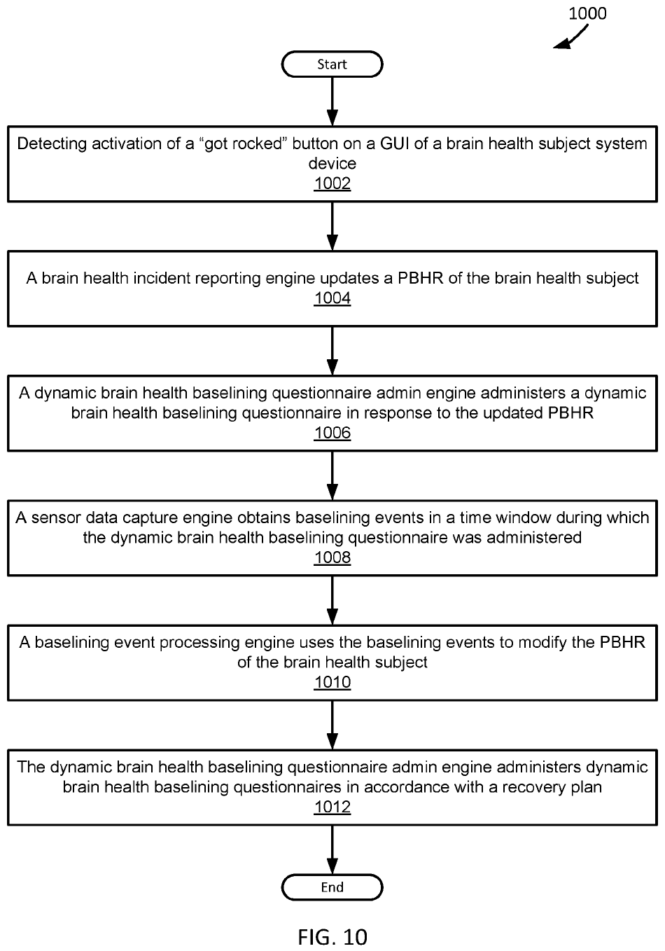

FIG. 10 depicts a flowchart 1000 of an example of a method for brain health incident reporting and monitoring during recovery. The flowchart 1000 begins at module 1002 with detecting activation of a “got rocked” button on a graphical user interface (GUI) of a brain health subject system device. An applicable engine such as the brain health incident reporting engine 332 in FIG. 3 provides an interface for a brain health subject to indicate a brain health incident has occurred.

The flowchart 1000 continues to module 1004 with a brain health incident reporting engine updates a PBHR of a brain health subject. An applicable datastore such as the brain health baseline record datastore 316 of FIG. 3 stores the PBHR.

The flowchart 1000 continues to module 1006 with a dynamic brain health baselining questionnaire admin engine administers a dynamic brain health baselining questionnaire in response to the updated PBHR. An applicable engine such as the dynamic brain health baselining questionnaire admin engine 314 administers the dynamic brain health baselining questionnaire when a persona of a brain health subject matches a parameters of the dynamic brain health baselining questionnaire.

The flowchart 1000 continues to module 1008 with a sensor data capture engine obtains baselining events in a time window during which the dynamic brain health baselining questionnaire was administered. An applicable engine such as the sensor data capture engine 320 of FIG. 3 captures sensor data, some of which is captured during a time window during which a dynamic brain health baselining questionnaire is administered.

The flowchart 1000 continues to module 1010 with a baselining event processing engine uses the baselining events to modify the PBHR of the brain health subject. An applicable engine such as the baselining event processing engine 328 CRUDs the brain health record datastore 316, which can be characterized as a PBHR of the brain health subject, using the baselining events.

The flowchart 1000 ends at module 1012 where the dynamic brain health baselining questionnaire admin engine administers dynamic brain health baselining questionnaires in accordance with a recovery plan. The dynamic brain health baselining questionnaire admin engine 314 of FIG. 3 administers persona-specific dynamic brain health baselining questionnaires, and a brain health subject who is recovering from a brain health incident can be characterized as having a persona. Questionnaires can be provided to monitor recovery milestones.

These and other examples provided in this paper are intended to illustrate but not necessarily to limit the described implementation. As used herein, the term “implementation” means an implementation that serves to illustrate by way of example but not limitation. The techniques described in the preceding text and figures can be mixed and matched as circumstances demand to produce alternative implementations.

1. A method comprising:
generating a dynamic brain health baselining questionnaire;
providing the dynamic brain health baselining questionnaire to a brain health subject system;
obtaining a brain health baseline record for a brain health subject from the brain health subject system;
receiving a brain health baseline request for the brain health subject by an authorized system;
providing a brain health baseline record of the brain health subject to a subscriber system.

2. The method of claim 1, wherein the generating is in accordance with a plurality of parameters for determining a resulting question for a specific brain health subject, referencing a PBHR of the specific brain health subject for that purpose, or in accordance with a plurality of parameters for determining a resulting question for a specific persona.

3. The method of claim 1, wherein the generating is in accordance with a plurality of parameters with parameter values that are modified through machine learning techniques.

4. The method of claim 1, wherein the questionnaire includes a first chain of questions generated for first one or more brain health subjects having a first attribute and a second chain of questions is generated for second one or more brain health subjects having a second attribute.

5. The method of claim 1, wherein the questionnaire includes a first question generated in response to a first response or reaction to a previous question and a second question is generated for a second response or reaction to the previous question.

6. The method of claim 1, wherein the dynamic brain health baselining questionnaire is provided to the brain health subject system when the PBHR identifies a persona matching a defined persona of the dynamic brain health baselining questionnaire.

7. The method of claim 1, wherein the dynamic brain health baselining questionnaire is provided to the brain health subject system when the PBHR identifies a timing matching a window of the dynamic brain health baselining questionnaire.

8. The method of claim 1, wherein the dynamic brain health baselining questionnaire is provided to the brain health subject system in a push message at a predetermined time or after a triggering event for brain health subjects identifiable as having a persona matching that of the dynamic brain health baselining questionnaire.

9. The method of claim 1, wherein the dynamic brain health baselining questionnaire is provided to the brain health subject system in a push message in advance of a predetermined time or predicted triggering event.

10. The method of claim 1, wherein dynamic brain health baselining questionnaire is provided to the brain health subject system in a pull message in response to requests from the brain health subject system.

11. The method of claim 1, wherein the dynamic brain health baselining questionnaire is provided to the brain health subject system in response to a request that includes one or more profile attributes of a brain health subject associated with the brain health subject system.

12. The method of claim 1, comprising obtaining profile attributes from the PBHR datastore to enable provisioning of a dynamic brain health questionnaire.

13. The method of claim 1, comprising:
obtaining a request for the dynamic brain health baselining questionnaire from the brain health subject system;
retrieving a brain health baselining questionnaire corresponding to profile attributes of the brain health subject by referring to a brain health baselining questionnaire table.

14. The method of claim 1, wherein:
the dynamic brain health baselining questionnaire is a first dynamic brain health baselining questionnaire;
the brain health baseline record includes brain health baselining inputs made by a brain health subject in response to a second dynamic brain health baselining questionnaire.

15. The method of claim 1, comprising generating brain health data statistics based on one or more brain health baseline records of multiple subjects.

16. The method of claim 1, comprising adding brain health baselining information obtained in association with a subject to a PBHR associated with the subject, resulting in a new brain health baseline for the subject.

17. The method of claim 1, comprising:
updating a personal brain health record (PBHR) for the brain health subject in a PBHR datastore using the brain health baseline record from the brain health subject system;
providing the brain health baseline record of the brain health subject from the PBHR datastore to the authorized party.

18. The method of claim 1, receiving one or more attributes unique to a subscriber associated with the authorized party and to a brain health baseline record.

19. The method of claim 1, wherein the authorized system and the subscriber system are the same system.

20. A computer program product including a memory and one or more processors for executing instructions in the memory to:
generate a dynamic brain health baselining questionnaire;
provide the dynamic brain health baselining questionnaire to a brain health subject system;
obtain a brain health baseline record for a brain health subject from the brain health subject system;
receive a brain health baseline request for the brain health subject by an authorized system;
provide a brain health baseline record of the brain health subject to a subscriber system.
